SEQUENCE LISTING (2 sequences)

SEQ ID NO: 1 - protein, 461 aa, human poliovirus 1
GEIQWMRPSKEVGYPIINAPSKTKLEPSAFHYVFEGVKEPAVLTKNDPRLKTDFEEAIFSKYVGNKITEVDEYMKEAVDHYAGQLMSLDINTEQMCLEDAMYGTDGLEALDLSTSAGYPYVAMGKKKRDILNKQTRDTKEMQKLLDTYGINLPLVTYVKDELRSKTKVEQGKSRLIEASSLNDSVAMRMAFGNLYAAFHKNPGVITGSAVGCDPDLFWSKIPVLMEEKLFAFDYTGYDASLSPAWFEALKMVLEKIGFGDRVDYIDYLNHSHHLYKNKTYCVKGGMPSGCSGTSIFNSMINNLIIRTLLLKTYKGIDLDHLKMIAYGDDVIASYPHEVDASLLAQSGKDYGLTMTPADKSATFETVTWENVTFLKRFFRADEKYPFLIHPVMPMKEIHESIRWTKDPRNTQDHVRSLCLLAWHNGEEEYNKFLAKIRSVPIGRALLLPEYSTLYRRWLDSF

SEQ ID NO: 2 - protein, 462 aa, Coxsackievirus
GEIEFIESSKDAGFPVINTPSKTKLEPSVFHQVFEGNKEPAVLRSGDPRLKANFEEAIFSKYIGNVNTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSIEKVAKGKSRLIEASSLNDSVAMRQTFGNLYKTFHLNPGVVTGSAVGCDPDLFWSKIPVMLDGHLIAFDYSGYDASLSPVWFACLKMLLEKLGYTHKETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNATFLKRYFRADEQYPFLVHPVMPMKDIHESIRWTKDPKNTQDHVRSLCLLAWHNGEHEYEEFIRKIRSVPVGRCLTLPAFSTLRRKWLDSF

(19) United States
(12) Patent Application Publication
(10) Pub. No.: US 2010/0196874 A1
(43) Pub. Date: Aug. 5, 2010

(54) METHOD OF DRUG DESIGN

(75) Inventors: Elena V. Gazina, Victoria (AU); Steven Petrou, Victoria (AU); David N. Harrison, Victoria (AU); David A. Anderson, Victoria (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: PICORAL PTY LTD, Melbourne, Victoria (AU)

(21) Appl. No.: 12/519,018

(22) PCT Filed: Dec. 13, 2007
(86) PCT No.: PCT/AU07/01930

§ 371 Date: Mar. 30, 2010

(30) Foreign Application Priority Data
Dec. 13, 2006 (AU) 2006906948

(51) Int. Cl. C12Q 1/70; C12Q 1/48; C12N 7/01; G06F 17/50

(52) U.S. Cl. 435/5; 435/15; 435/235.1; 703/1

(57) ABSTRACT

The description discloses that amiloride-like compounds inhibit enterovirus RNA replication by interaction with RNA dependent RNA polymerase (RdRP). The description discloses in silico and in vitro methods of screening for inhibitors of RdRP activity, amiloride-resistant enterovirus variants and amiloride-like compounds.

### BACKGROUND

1. Field

The present invention relates generally to methods of designing and testing anti-viral drugs. In particular, the present invention relates to the development of amiloride-like compounds. In another aspect, the present invention relates to the use of molecular models generated by a computer to design agents that associate with an RNA dependent RNA polymerase (RdRP) such as, without limitation, an RdRP from a member of the Picornaviridae.

2. Description of the Prior Art

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Pyrazine derivatives such as amiloride (3,5-diamino-6-chloro-N-(diaminomethylidene) pyrazine-2-carboxamide) and EIPA 5-(N-ethyl-N-isopropyl)amiloride are known as ion-channel inhibitors and have previously been shown to inhibit the replication of representative members of the Rhinovirus genus within the Picornaviridae family (International Publication No. WO 03/063869 incorporated herein in its entirety; Gazina et al., Antiviral Res. 67:98-106, 2005). This antiviral effect was shown to be due to inhibition of both intracellular virus replication, and the release of progeny virus from the cell, both of which result in a reduced level of virus infection in subsequent rounds of cell infection.

Ion channels are encoded in the genomes of many viruses, including the vpu protein of human immunodeficiency virus (HIV), the M protein of Dengue virus, the E protein of Coronavirus, and the p7 protein of hepatitis C virus. There are no identified ion channel proteins encoded within the Picornavirus genome (approximately 7.5 kb single strand, positive sense RNA), however it is well known that Picornaviruses recruit cellular proteins into virus-induced replication complexes during their intracellular replication. As such, the antiviral effect of these compounds was considered to be most likely through their effect on either (i) a previously unidentified viral ion channel protein encoded by the Picornaviruses, or (ii) an ion channel protein encoded by the cell, which was in turn involved in Picornavirus replication.

There is a need to understand the mechanism of anti-viral compounds inter alia to facilitate the rational design of new anti-viral agents.

### SUMMARY

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Each embodiment described herein is to be applied mutatis mutandis to each and every other embodiment unless specifically stated otherwise.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided after the claims.

In a broad aspect, the present invention provides a method of anti-viral drug design or testing comprising the use of structural coordinates comprising an interacting site of a viral RNA dependent RNA polymerase (RdRP) and/or a variant thereof and/or a viral RNA dependent RNA polymerase (RdRP) activity assay to evaluate the anti-viral activity of amiloride or EIPA and/or an amiloride or EIPA derivative and/or amiloride-like compounds. The method employs any suitable measure of RdRP activity. The term “activity” includes level of level or amount of RdRP produced as well as the functional activity such as enzymatic activity or binding. In one non-limiting embodiment the activity assay evaluates RdRP amount, binding or enzymatic activity. One convenient assay is a high throughput RNA polymerase assay. In an exemplified embodiment the RdRP is an enterovirus RdRP.

Amiloride-like compounds which are active against viral RdRP the compounds are also tested for their anti-viral activity against other virus and virus subtypes selected from within the picornaviridae such as enterovirus or rhinovirus against which amiloride and amiloride derivatives are know to be active. They may also be tested against hepatovirus (hepatitis A virus). In some embodiments compounds are selected which are active against enteroviruses and rhinoviruses and/or heptatoviruses (these being the three picornavirus genera which cause significant disease). Such compounds would be expected to show synergy with existing antivirals that do not target RdRP and thus combination therapy including the use of agents that affect different targets also forms part of the present invention.

By “amiloride-like compounds” is meant those compounds that have the functional capacity of amiloride or its functional derivatives as disclosed herein to bind to and modify RdRP. These molecules include mimetics of amiloride or its derivatives that show structural similarity with amiloride or amiloride derivatives only to the extent sufficient to interact with RdRP. In some embodiments, the amiloride-like compound is selected from the group consisting of amiloride (3,5-diamino-6-chloro-N-(diaminomethylidene) pyrazine-2-carboxamide), EIPA (5-(N-ethyl-N-isopropyl)amiloride), Benzamil, and HMA (5-(N,N-hexamethylene)amiloride) or a derivative or variant. International Publication No. WO 03/063869 incorporated herein in its entirety discloses amiloride-like compounds and amiloride derivatives in accordance with the present invention. These molecules are the starting point in the screening and development of new anti-RdRP agents. The preparation and derivation of amiloride derivatives and variants is known to those of skill in the art or medicinal chemistry.

In the preparation of amiloride-like compaounds and amiloride derivatives for use as antiviral agents, it is preferable in some embodiments to minimize the ion channel activity of the compounds so as to minimize the potential for side effects in a patient subject due to such activity, while maximizing the antiviral activity. Table 7 sets out the relative antiviral potency and toxicity of a range of amiloride derivatives, as tested in HeLa cell culture with CVB3. The toxicity of the compounds (CC50, being the concentration causing 50% reduction in cell metabolism as measured by Alamar Blue assay) reflects the relative activity of each compound against cellular ion channels, while the antiviral activity (IC50) reflects the relative activity of each compound against the RdRP of CVB3. It is evident that activity against cellular ion channels and activity against RdRP are not proportionally linked, with some examples such as CMPG and DMA (highlighted) showing essentially no specific antiviral activity above ion channel toxic activity (IC50/CC50 ratio less than 2.5), whereas other compounds such as Amiloride and CHG (highlighted) have IC50/CC50 of more than 18, and EIPA, HMA and others have IC50/CC50 of between 2.5 and 18. This demonstrates that it is possible to prepare and derive amiloride derivatives and variants in a way that can maximize antiviral activity and minimize ion channel activity, including the systematic study of structure-activity relationships (SAR) for such derivatives as well known in the art.

In some embodiments, the method is directed to developing new anti-viral compounds. In other embodiments, the method is aimed at testing the ability of amiloride-like compounds capable of binding to RdRP to inhibit viral replication or affect viral RdRP activity in a sample derived from an infected subject. Thus, in this latter embodiment, a sample from the subject comprising viral particles is directly or indirectly tested for viral resistance or an effect on viral RdRP activity in the presence of one or more anti-viral compound.

Accordingly, in one embodiment the present invention provides a method of anti-viral drug testing comprising the use of a viral RNA dependent RNA polymerase (RdRP) activity assay to evaluate the anti-viral activity of amiloride or EIPA and/or an amiloride or EIPA derivative and/or amiloride-like compounds.

In some embodiments, RdRP binding molecules are identified in in silico docking screens using picornavirus RdRP, such as polio 3D and CVB3 RdRP. In some embodiments, the invention provides a method of evaluating the ability of a test compound to modulate viral activity wherein said method comprises: computationally generating a three dimensional molecular representation comprising at least one interacting site of a viral RdRP and/or a variant thereof; computationally generating a three dimensional molecular representation of the test compound; performing a molecular fitting (docking) operation; computationally quantifying the association between the RdRP and/or a variant thereof and the test compound based on the output of the fitting (docking) operation. In some embodiments the molecular representation includes an interacting site of a viral RdRP bound to GTP. In other embodiments, the molecular representation includes an interacting site of a viral RdRP bound to NTP. In some embodiments, the interacting site comprises one or more of a palm domain and a finger domain (pinky, middle, ring, index and/or thumb). In some embodiments, the palm domain comprises one or more or consists of polymerases domains including: motif A (aa225-240 of 3D of poliovirus or corresponding amino acids from other Picornaviridae); motif B (aa290-312 or corresponding amino acids from other Picornaviridae); motif C (aa318-336 or corresponding amino acids from other Picornaviridae); motif D (339-354 or corresponding amino acids from other Picornaviridae); motif E (aa369-380 of 3D of poliovirus, aa370-381 of CVB3 or corresponding amino acids from other Picornaviridae).

In some embodiments, the interacting site of RdRP comprises an NTP-binding centre and/or an E motif. In some embodiments, the three dimensional molecular representation of RdRP consists essentially of an NTP-binding centre and/or an E motif.

In another embodiment, the method comprises introducing the an compound into a viral RNA dependent RNA polymerase (RdRP) activity assay and evaluating the ability of said compound to modulate RdRP activity based on the output from the activity assay.

In accordance with some embodiments of the present method, the RdRP is a picornavirus RdRP and/or a RdRP-like variant thereof. The molecular representation of the picornavirus RdRP may be derived in some embodiments from the atom coordinates of Polio RdRP, for example, by molecular replacement. The molecular coordinates of polio RdRP are described in Hansen et al. in Structure 5:1109-22, 1997; Thompson et al., 2004 (supra) and publicly available databases, such as the Protein Database (PDB) and corrected versions thereof.

Illustrative picornaviruses are selected from those belonging to a genus selected from Enterovirus, Rhinovirus, Hepatovirus, Cardiovirus and Aphthovirus. In some embodiments, the picornavirus is a polio RdRP-like variant comprising an interacting site whose root mean square deviation from the structure coordinates of the Cα atoms of polio RdRP is not more than about 1.0 Å to about 1.5 Å. In other embodiments, the picornavirus is a Coxsackievirus RdRP comprising an interacting site whose root mean square deviation from the structure coordinates of the Cα atoms of polio RdRP is not more than about 1.0 Å to about 1.5 Å.

In some embodiments, the variant is modelled from polio or other available structural coordinates for polymerases using the primary amino acid sequence of a picornavirus selected from Enterovirus, Rhinovirus, Hepatovirus, Cardiovirus, Aphthovirus, Parechovirus, Erbovirus, Kobovirus and Teschovirus.

In other embodiments, the RdRP variant is an amiloride resistant mutant form of RdRP as described herein. In other embodiments, the variant is a precursor compound or functional part, derivative or homolog. The present invention extends to amiloride-resistant CVB3 variants comprising a modified RdRP. In an exemplified embodiment, the variants are modified close to the active centre of RdRP such as further disclosed herein. In a further exemplified embodiment the variants comprise one or two or more RdRP mutations including S299T, A372V and/or D48G.

The compounds contemplated by the present invention encompass molecules such as a synthetic or naturally occurring compounds, a peptide, peptidomimetic, or a pharmaceutical composition. In some embodiments, the compound inhibits RdRP enzyme activity. In some embodiments, the subject compounds are amiloride compounds or derivatives such as, but in no way limited to, those described in International Publication No. WO 03/063869. The skilled addressee will appreciate that given the presently disclosed information a wide range of amiloride or EIPA derivatives can be fashioned to interact with RdRP.

Another aspect of the present invention contemplates compounds identified in the herein described methods. In some embodiments, the compound interacts with the palm or finger domain of RdRP. In other embodiments, the compound interacts with the E motif of RdRP or the NTP-binding motif of RdRP. The invention has been so far illustrated using RdRP from coxsackievirus. However, the invention extends to any structurally similar RdRP preferably selected from a member of the picornavirus family.

The present invention also provides a method of inhibiting picornavirus replication comprising administering an inhibitor of RdRP identified by any one of the subject methods. In a preferred embodiment the picornavirus is an enterovirus.

In some embodiments, the use of amiloride-like compounds are described herein is contemplated in the manufacture of a medicament for the treatment or prevention of a picornavirus infection. The term “manufacture” includes selection or design of a medicament. In other embodiments, a process is provided for making a compound that inhibits RdRP, comprising carrying out one of the herein described methods to identify a compound or amiloride-like compound; and manufacturing the compound according to methods known in the art.

Accordingly, the use of an amiloride-like compound in a process for identifying inhibitors of an RdRP is proposed. The description provides a method for identifying a compound which inhibits RdRP activity, the method comprising contacting in silico or in vitro an RdRP and/or a variant thereof with an amiloride-like compound and determining whether or not an activity of RdRP is decreased in the presence of the amiloride-like compound. In some embodiments, The RdRP activity is RdRP binding or RdRP enzymatic activity.

Screening assays based upon competitive screens are contemplated and in another embodiment, the description provides a method for identifying a compound which inhibits RdRP activity, the method comprising contacting an RdRP and/or variant thereof with a competitor amiloride-like compound wherein said competitor comprises a detectable label, whereby said competitor binds to RdRP and/or a variant thereof and is capable of being displaced by an inhibitor. In some embodiments, the RdRP is an enterovirus RdRP and/or a variant thereof. In an exemplified embodiment, the enterovirus is poliovirus or coxsackievirus. Amiloride resistant RdRPs are usefully employed to probe structure-function activities and in some embodiments the RdRP variant is an amiloride resistant mutant form of RdRP such as are disclosed herein.

In another embodiment the invention contemplates a method of drug design comprising selecting a molecule which is an amiloride derivative or an amiloride-like compound wherein said molecule inhibits the biological activity of RdRP, said method comprising: selecting said molecule on the basis of enhanced binding to RdRP and in some particular embodiments, enhanced binding to a palm or finger domain of picornavirus RdRP.

In yet another embodiment, the present invention provides a method of treatment of a subject having a picornavirus infection, said method comprising administering an effective amount of a compound identified according to any one of the subject methods herein disclosed for a time and under conditions sufficient to treat the subject.

This summary is not and should not be seen in any way as an exhaustive recitation of all embodiments of the present disclosure.

### BRIEF DESCRIPTION OF THE FIGURES

Some figures contain color representations or entities. Colored versions of the figures are available from the Patentee upon request or from an appropriate Patent Office. A fee may be imposed if obtained from a patent office.

FIG. 1 is a graphical representation showing the antiviral effect (expressed as % virus yield compared to untreated cultures) and the cytotoxic effect (expressed as % cell metabolism in the Alamar Blue assay compared to untreated cells) for amiloride, EIPA, benzamil and HMA against CVB3 in HeLa cells. Each of the drugs showed a selective antiviral effect against CVB3, as shown previously for rhinoviruses.

FIG. 2 is a graphical representation showing the amount of virus produced by cultures which were infected with CVB3 (multiplicity of 1 plaque forming unit per cell), where 400 μM amiloride or 25 μM EIPA was added to the cultures at 1 hour intervals. Cultures were incubated until 10 hours post-infection, then the total virus yield (released virus+intracellular virus) was quantitated (FIG. 2A). Kinetics of virus production was measured in parallel (FIG. 2B). The results demonstrate that amiloride and EIPA are fully effective in reducing virus replication when added to infected cells up to 2 hours post-infection, which indicates that they do not have a significant effect on the binding, entry or uncoating of the virus. This contrasts with well-known antiviral drugs active against picornaviruses such as Pleconaril, which prevent uncoating and/or binding to the cell. In addition, the kinetics by which amiloride and EIPA continue to inhibit replication closely follows the kinetics of virus replication, which suggests that these drugs inhibit an intracellular replication event.

FIG. 3 is a graphical representation showing the proportion of virus released from cells infected with CVB3 and treated with amiloride or EIPA, added at various times as in FIG. 2, compared to untreated controls (NC). Both amiloride and EIPA resulted in a slight increase in the proportion of extracellular virus, in contrast to the reduced level of virus release previously reported for these drugs with Rhinoviruses (Gazina et al., 2005 (supra)). This suggests that the stage of virus replication affected by these drugs is between virus uncoating and virus release.

FIG. 4 is a graphical representation showing that the amount of virus produced in cells is directly proportional to the amount of viral RNA produced. Infected cells treated with 25 micromolar EIPA or with 500 micromolar guanidine produced equivalent amounts of viral RNA, and equivalent amounts of virus, suggesting that EIPA does not affect virus assembly. Infected cells treated with 400 micromolar amiloride showed greatly reduced levels of viral RNA by 3H-uridine labelling, despite having similar amounts of virus to cultures treated with EIPA. However, this appears to be due to indirect effects of amiloride on the ability of the cell to take up uridine from the culture medium, and is consistent with amiloride having no effect on virus assembly.

FIG. 5 is a photographic representation showing that the amount of viral protein synthesised in the presence of the drugs is not reduced significantly. This suggests that once the viral messenger RNA is produced through the process of RNA replication the drugs have no effect on translation of the viral RNA into viral proteins.

FIG. 6 is a graphical representation showing that the amount of viral RNA synthesised in the presence of the drugs is significantly reduced. FIG. 6A shows the kinetics of viral RNA replication, indicating that the peak of viral RNA replication under these conditions occurs between 4 and 5 hours post infection. FIG. 6B is a photographic representation showing that when infected cells are treated with amiloride or EIPA during this time, the amount of viral RNA synthesis is dramatically reduced compared to untreated cells. This is similar to the reduction observed in guanidine treated cells, where guanidine is known to directly inhibit viral RNA replication. These results suggest that amiloride and EIPA have a direct effect on viral RNA replication.

FIG. 7 is a graphical representation showing that all six clones passaged in the presence of amiloride show reduced inhibition of virus replication in the presence of drug, compared to wild-type virus. These results demonstrate that mutation(s) in viral proteins are sufficient to overcome the antiviral effects of amiloride, suggesting that the drug may be acting directly on a viral protein rather than on a cellular protein that is recruited by the virus. However it should be noted that picornaviruses are also able to develop resistance to some drugs such as brefeldin A, which act on cellular proteins, by the acquisition of mutations that allow the virus to use redundant biochemical pathways in the cell rather than the brefeldin-sensitive pathway.

FIG. 8 is a graphical representation showing that the six clones of amiloride-resistant virus remained sensitive to the antiviral effects of guanidine, and that six clones of guanidine-resistant virus (prepared by sequential passaging in the presence of guanidine) remained sensitive to the antiviral effects of amiloride. These results indicate that amiloride and guanidine have different mechanisms of antiviral action, despite sharing the structural similarity of the guanidine group.

FIG. 9 is a graphical representation showing a schematic representation of the genome and encoded proteins of CVB3. CVB3 encodes a single polyproteins which is then cleaved by viral proteases to yield at least eleven different viral proteins which are nominally assigned to three regions. The P1 region contains the viral capsid structural proteins, VP1, VP2, VP3 and VP4; because amiloride did not affect viral attachment, uncoating or release it was considered unlikely that drug-resistance mutations would be found in this region, and it was not sequenced. The P2 region contains non-structural proteins involved in viral replication, 2A, 2B and 2C. 2B is a membrane-spanning protein but does not have any other resemblance to known ion channel proteins. 2C is a membrane-spanning protein that functions to associate the 3AB protein with the membranous replication complex, and is the target for inhibition of RNA replication by guanidine. The P3 region contains further non-structural proteins involved in RNA replication; the 3A protein, the 3B protein (VPg) which acts as a primer for RNA replication; the 3C protein that is the virus-encoded protease, and the 3D protein that is the viral RNA-dependent RNA polymerase. The P2 and P3 regions of the genome were sequenced.

FIG. 10 tabulates the nucleotide and deduced amino acid sequences of the six amiloride-resistant clones. All isolates had a mutation within the 3D protein (viral RNA dependent RNA polymerase): S299T in three isolates, and A372V in the other three isolates. Four of six isolates had the D48G mutation in the 2A protein.

FIG. 11 is a graphical representation showing that viruses containing either S299T or A372V mutations show reduced sensitivity to both amiloride and EIPA, demonstrating that these amino acids are important in the mechanism of action of the drugs. In contrast, the D48G mutation, by itself, had no significant effect on the virus sensitivity to the drugs.

FIG. 12 is a representation of an alignment of Picornavirus RdRP amino acid sequences compared to the coxsackievirus CVB3 amino acid sequence. The BLASTP 2.2.15 [Oct. 15, 2006] was conducted as described for example in Altschul et al., Nucleic Acids Res., 25:3389-3402, 1997; Schaeffer et al., Nucleic Acids Res., 29:2994-3005, 2001. Using all non-redundant GenBank CDS translations+PDB+SwissProt+P1R+PRF excluding environmental samples and comprised 4,258,188 sequences; and 1,464,798,397 total letters,

FIG. 13 is an alignment of 3DPol amino acid sequence for Poliovirus type 1 (Mahoney, P03300) (SEQ ID NO: 1) and Coxsackievirus B3 (Nancy, P03313) (SEQ ID NO: 2). The two sequences show 75% identity and 85% similarity (the similarity measure ignores conservative substitutions) and one amino acid gap identified as “−”. A consensus sequence is shown between the Polio and CVB3 sequence where “+” indicates conservative amino acid differences. Amino acid sequences representing function domains are colored in Poliovirus sequence but apply to CVB3 sequences. Amino acid mutations shown to confer resistance to amiloride in CVB3 are shown in red, and highlighted in yellow background. Note that Poliovirus type 1 (Mahoney) has the A372V mutation of CVB3 as its normal sequence. Residue D238, which binds the NTP in place for polymerase function, is highlighted in green background. The GDD active site of the polymerase is highlighted in magenta background. The amino acid sequence comprising about amino acids 1 to 69 is the index domain. The amino acid sequence comprising about amino acids 96 to 149 is the pinky domain. The amino acid sequence comprising about amino acids 150 to 180 is the ring domain. The amino acid sequence comprising about amino acids 181 to 191 or 240 is the second part of the pinky domain. The amino acid sequence comprising about amino acids 269 to 286 is the middle domain. The amino acid sequence comprising about amino acids 327 to 329 is the GDD domain. The amino acid sequence comprising about amino acids 381 to 461 (382 to 462) is the thumb domain.

FIG. 14 is a representation of the molecular structure of poliovirus 3Dpol RdRp structure (taken from Thompson et al., EMBO J., 23:3462-3471, 2004). (A) Comparison of the original partial structure (yellow) with the complete structure shown with the fingers domain in red, the palm in gray, the thumb in blue, and the active site colored magenta. The N-terminal strand (residues 12; 36) of the original structure that descended toward the active site is shown in green. The two structures were superimposed using the backbone atoms of the active site GDD motif and three residues on either side of it (i.e. residues 324; 332). (B) Superimposition of the thumb domains from the original structure (yellow) and new complete structure (blue) showing that the thumb structure is largely unchanged by the two mutations (L446D and R455D) used to break Interface I and crystallize 3Dpol in a new lattice. The side chains of Phe30 and Phe34 are shown in green for the original structure and red for the new complete structure. (C) Top view of the complete 3Dpol structure highlighting the individual fingers of the fingers domain. The index finger is shown in green, the middle finger in orange, the ring finger in yellow, and the pinky finger in pink. As in (A), the palm is shown in gray, the thumb is in blue, and the active site is colored magenta. Phe30 and Phe34 are shown as sticks, Pro119 on the pinky finger is indicated with spheres, and glycines 117 and 124 are colored in cyan. (D) Bar representation of the 3Dpol sequence colored according to the structural elements shown in (C). Sections of the sequence in the palm are in gray and the numbers correspond to the first residue in a given structural motif.

FIG. 15 is a representation of an electron density map taken from Thompson et al., 2005 (supra). (B) Electron density map and model of the GTP molecule bound to 3Dpol with the 2′ OH group making a 2.8 angstrom long hydrogen bond with Asp238. The GTP makes bridging interactions between the fingers and palm domains. The base is staked on Arg174 from the ring finger, the ribose interacts with Arg174 from the ring finger and Asp238 in the palm, and the triphosphate interacts with Arg163 and Lys167 from the ring finger and the backbone of the palm domain.

Note the role of N297 (N298 in CVB3) in positioning D238 (D238 in CVB3) to interact with the NTP. The S299T amiloride resistance mutation suggests that amiloride binding may perturb the interaction of N298, providing an allosteric block to NTP binding.

### DETAILED DESCRIPTION OF THE INVENTION

Before describing the present invention in detail it is to be understood that unless otherwise indicated, the subject invention is not limited to specific formulations of components, screening methods, dosage regimens, or the like, as such may vary. It is also to be understood that the terminology used herein is for the purpose of describing particular embodiments only and is not intended to be limiting.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

Each embodiment described herein is to be applied mutatis mutandis to each and every other embodiment unless specifically stated otherwise.

The articles “a” and “an” are used herein to refer to one or to more than one (i.e. to at least one) of the grammatical object of the article. By way of example, “a compound” means one compound or more than one compound.

As used herein, the term “about” refers to a quantity, level, value, percentage, dimension, size, or amount that varies by as much as 30%, 20% or 10% to a reference quantity, level, value, percentage, dimension, size, or amount.

The term “agent” or “compound” “drug”, “medicament” “molecule”, “mimetic” and the like are used interchangeably herein to denote a compound that induces the desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to synthetic and naturally occurring molecules, proteinaceous molecules such as peptides, polypeptides and proteins as well as genetic molecules such as RNA, DNA and mimetics and chemical analogs thereof as well as cellular agents. These terms include combinations of two or more actives. A “combination” also includes a two-part or more such as a multi-part pharmaceutical composition where the agents are provided separately and given or dispensed separately or admixed together prior to administration.

The terms “effective amount” and “therapeutically effective amount” of an agent as used herein mean a sufficient amount of the agent to provide the desired therapeutic or physiological effect. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

“Analogs” contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogs.

The term ‘detectable label’ refers to any group that is linked to a competitor molecule such that when the competitor is associated with the RdRP target, the label allows recognition either directly or indirectly of the competitor such that it can be detected, measured and quantified. Examples of “detectable labels include photoreactive groups (such as benzophenones, azides and the like), fluorescent labels (including labels such as fluorescein, oregon green, dansyl, rhodamine, Texas red, phycoerythrin and Eu³⁺; reporter-quencher pairs include EDANS/DABCYL, tryptophan/2,4-dinitrophenyl, tryptophan/DANSYL, 7-methoxycoumarin/2,4-dinitrophenyl, 2-aminobenzoyl/2,4-dinitrophenyl and 2-aminobenzoyl/3-nitrotyrosine), chemiluminescent labels, colorimeteric labels, enzymatic markers, radioactive isotopes. Such labels are attached in a suitable position to the competitor by known methods. Suitable labelled competitor molecules are provided and are sold in kits for testing compounds that potentially bind to RdRP.

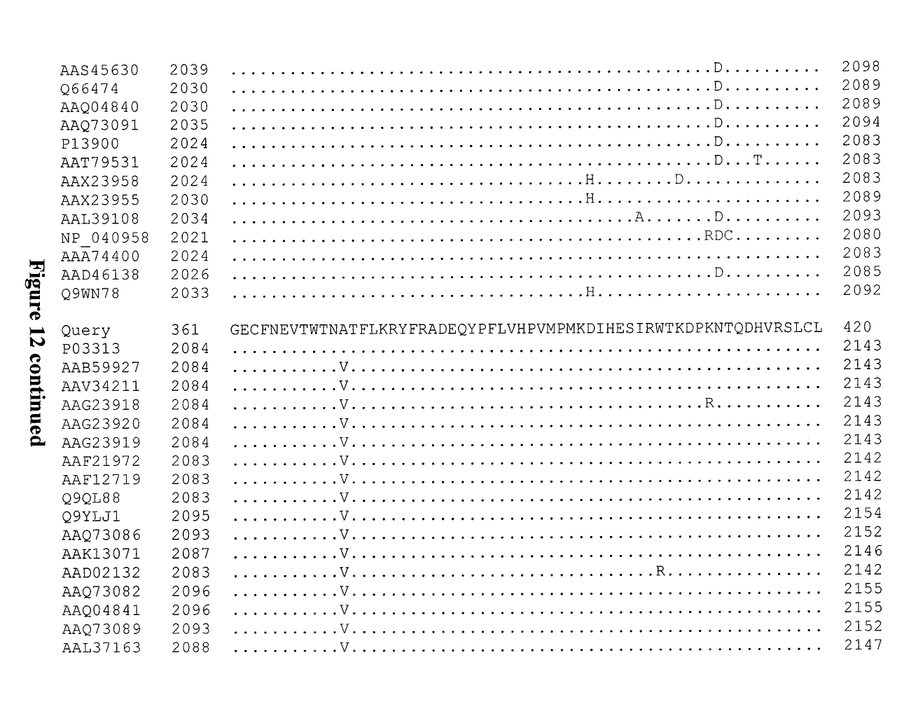

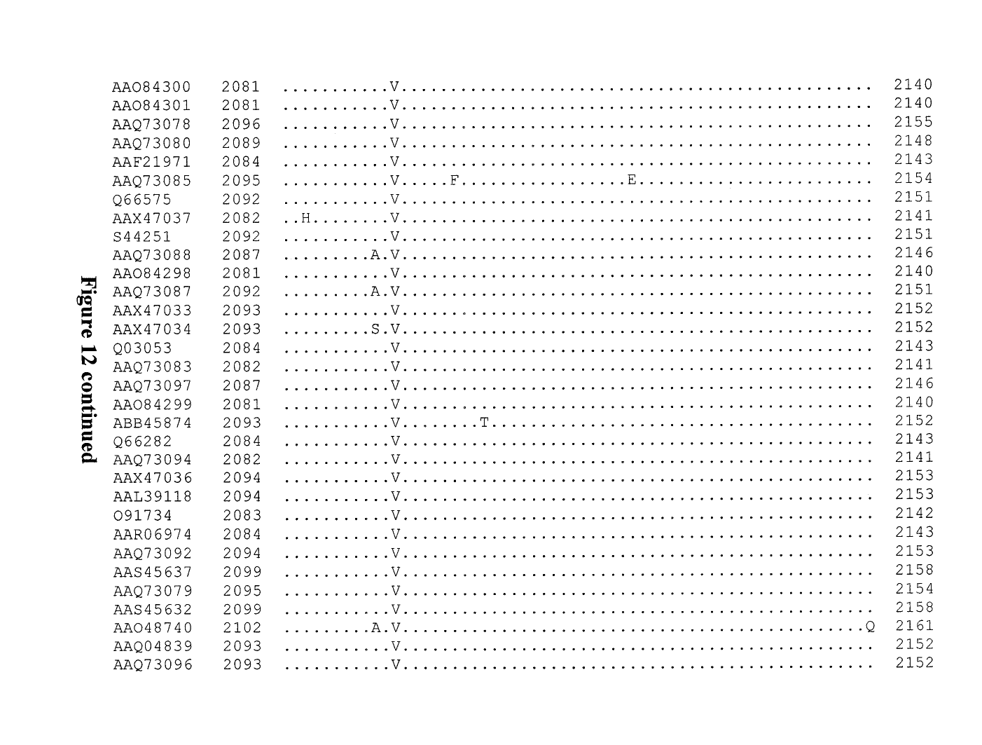

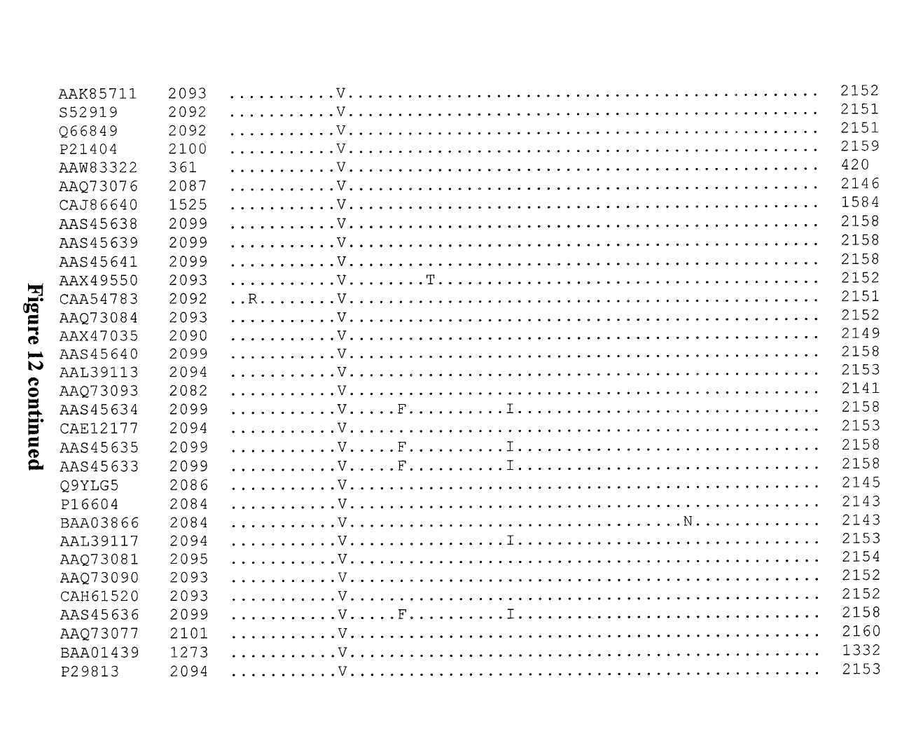

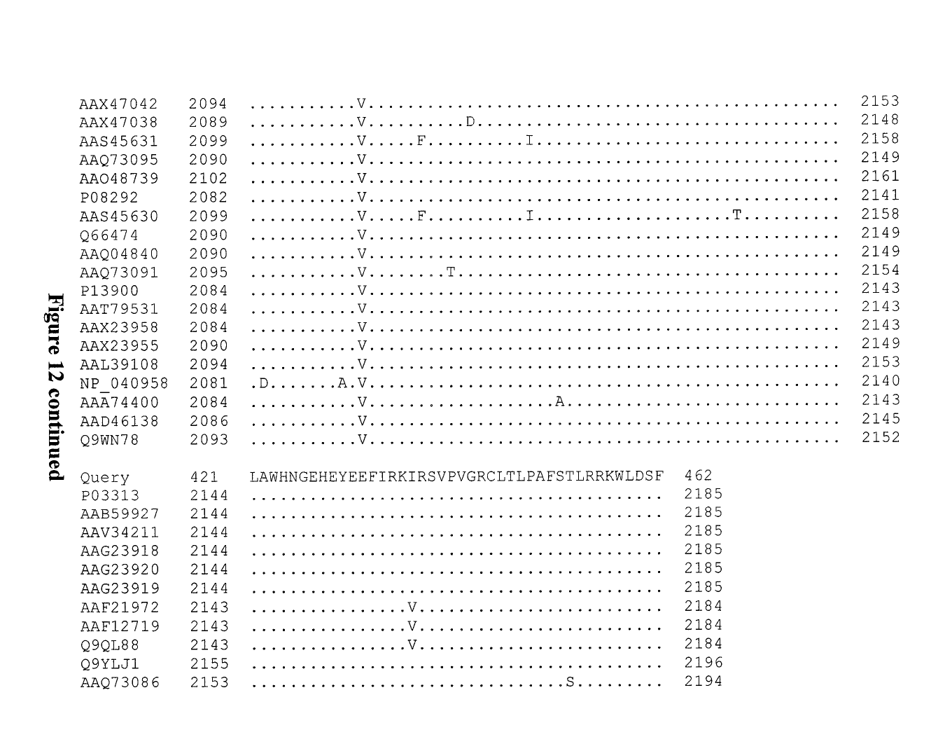

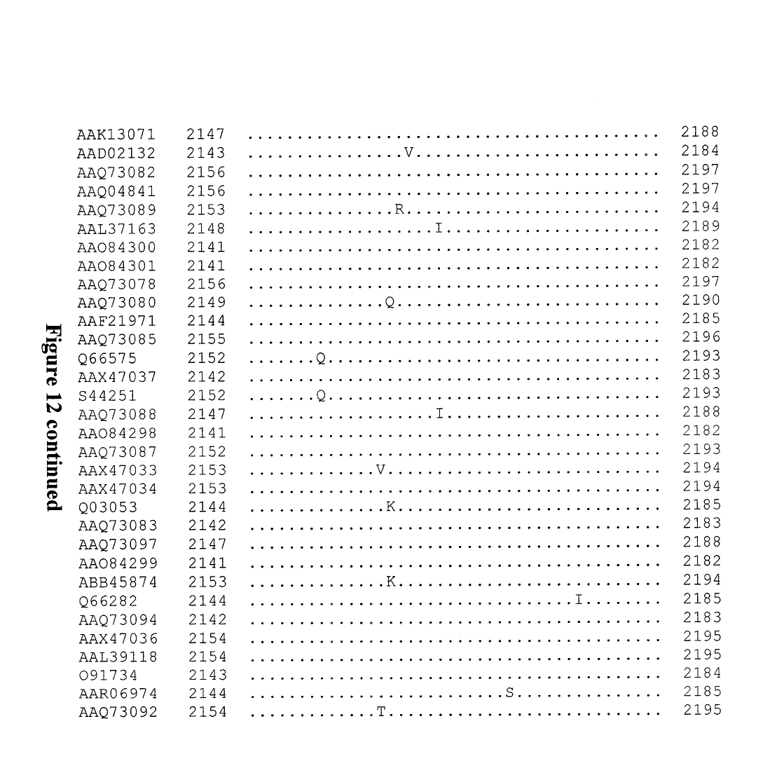

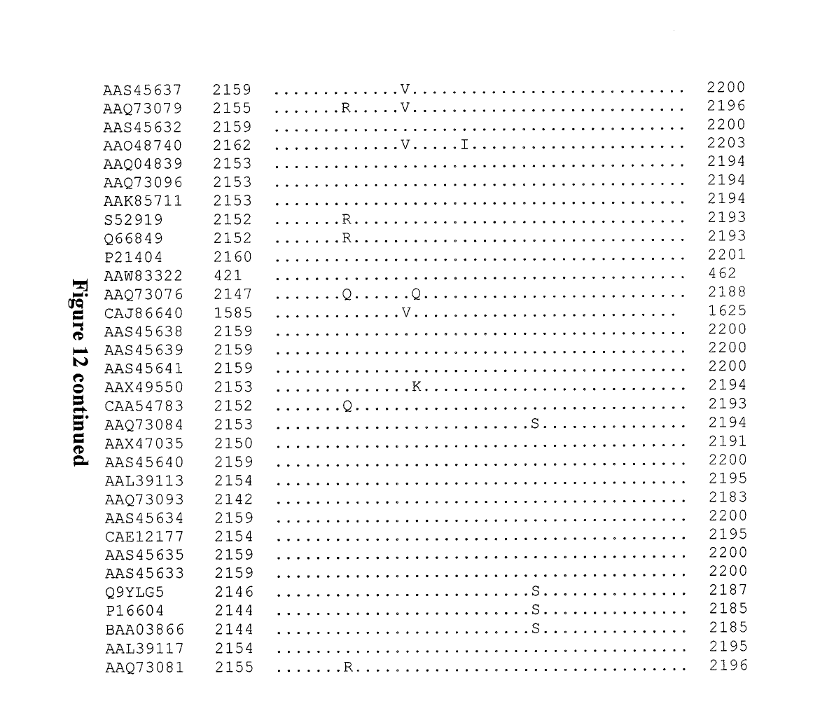

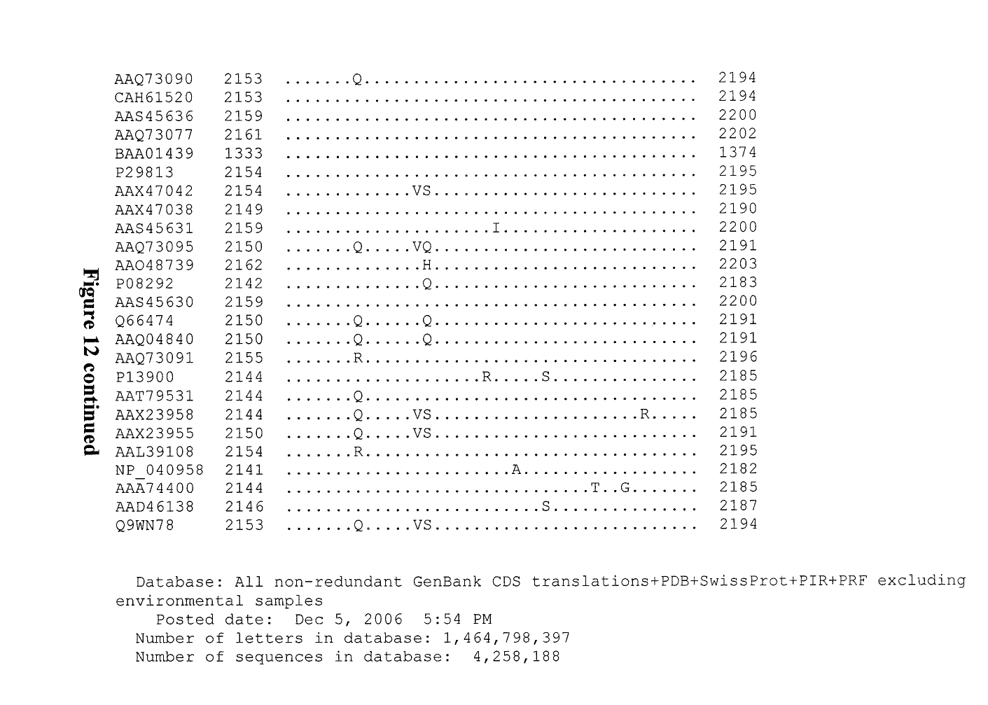

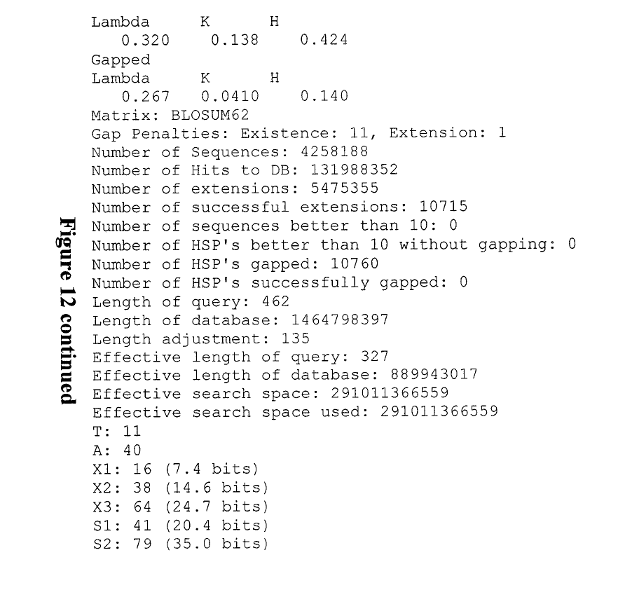

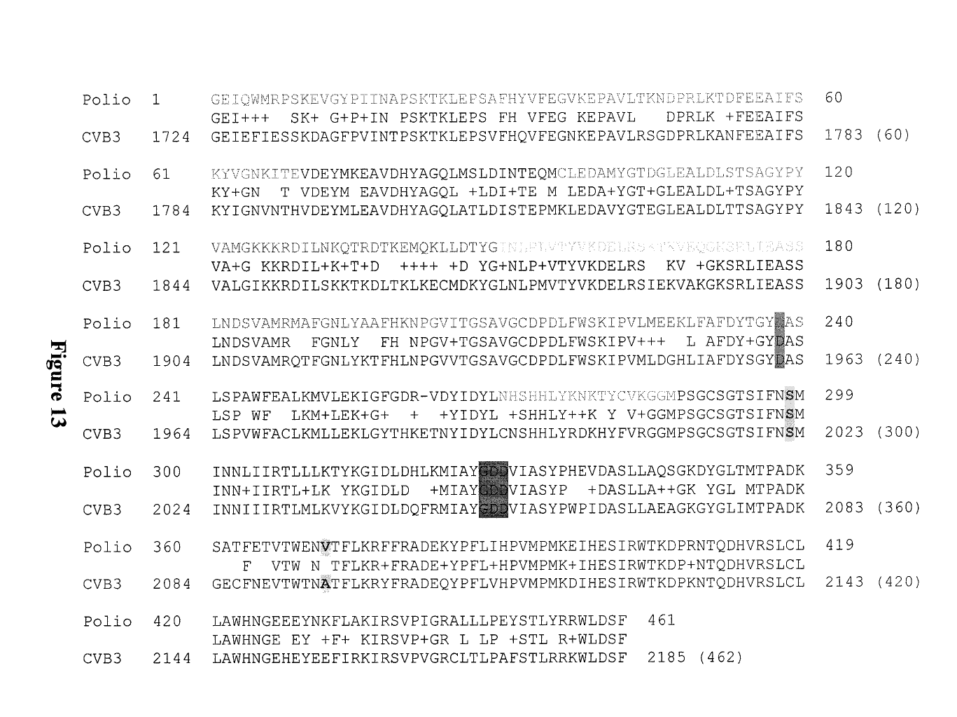

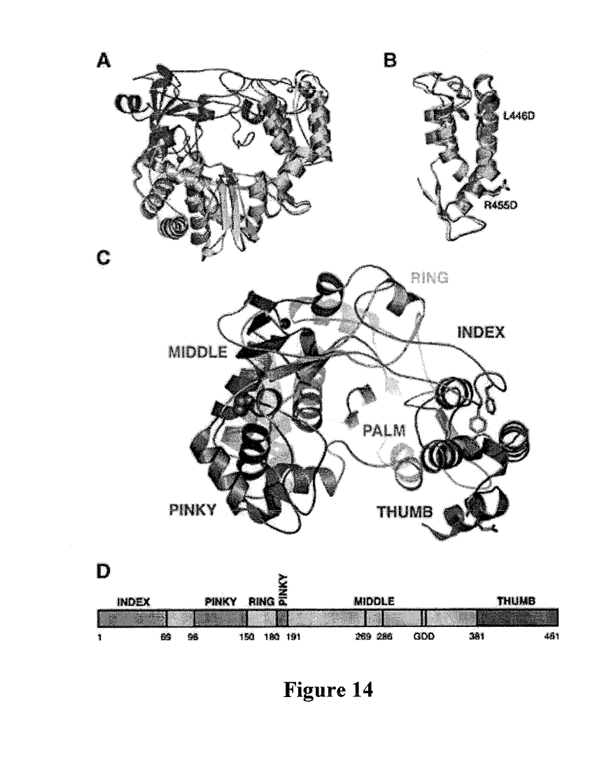

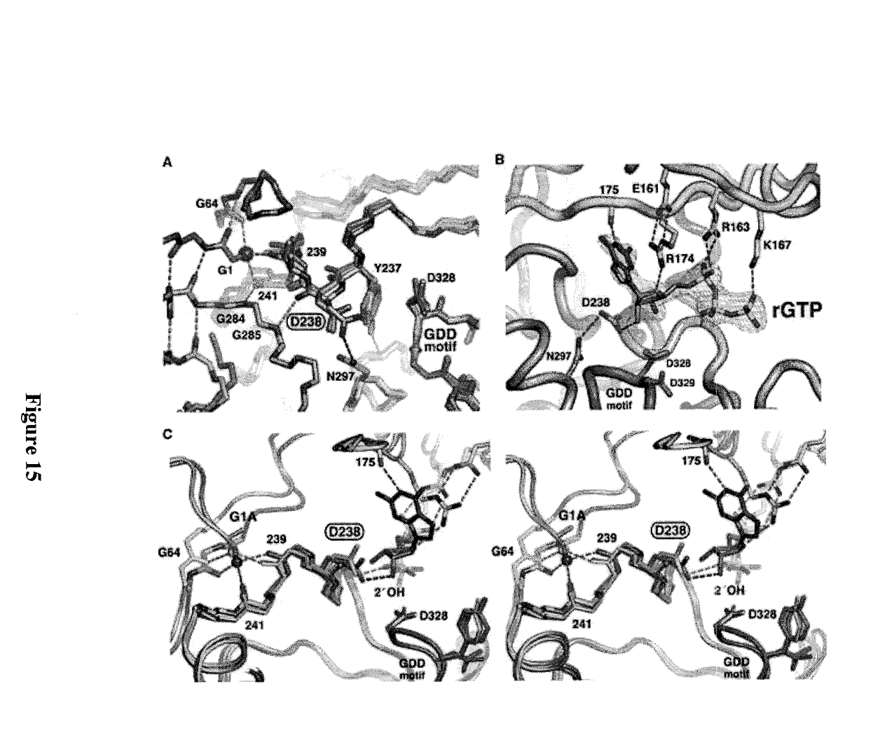

The present invention is predicated, in part, upon efforts to determine the mechanism by which various compounds exert their antiviral effect. Studies were conducted with the Picornavirus, Coxsackievirus B3 (CVB3), which is an important human pathogen having a high level of sequence identity with other members of the family. As shown in the Figures and Examples (see FIGS. 12 and 13), Picornavirus RdRPs are highly conserved and thus the present invention extends to methods employing RdRPs or variants thereof selected from other Picornavirus RdRPs such as those from the group comprising Enterovirus, Rhinovirus, Hepatovirus, Cardiovirus, Aphthovirus, Parechovirus, Erbovirus, Kobovirus and Teschovirus or variants of any one of these.

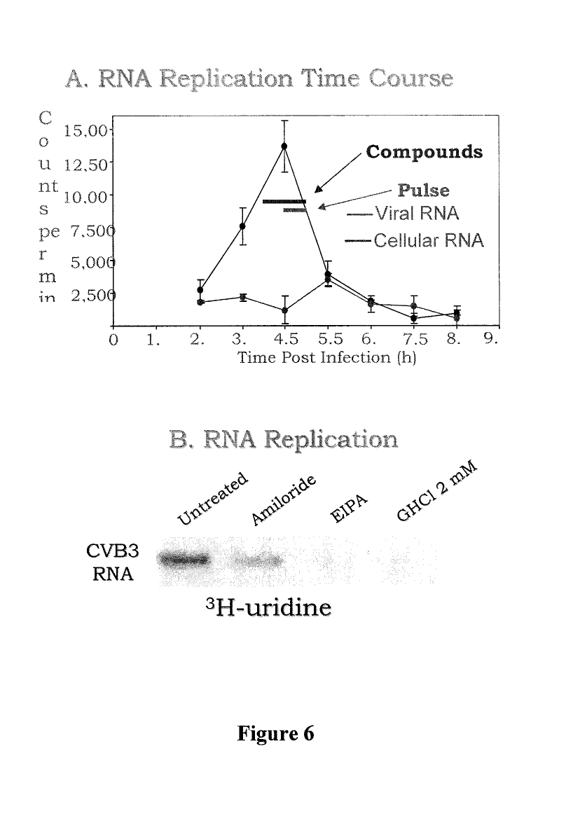

The replication of picornaviruses can be considered in the following stages: attachment to the cell; penetration of the cell; uncoating of the genome; translation of the polyproteins; proteolytic processing of the polyproteins; assembly of replication complexes; RNA replication; virus assembly; and virus release. The inventors have determined that pyrazine derivatives such as amiloride and its derivative EIPA have a direct effect upon viral RNA replication (see for example FIG. 6). Furthermore, the inventors have determined that mutations in the RdRP of Coxsackivirus cause amiloride resistance indicating that amiloride and its active derivatives are the first example of a non-nucleoside inhibitor of picornavirus RdRPs. In one aspect of the invention, this finding facilitates the design of a pharmacophore and lead structures and the screening of new anti-viral compounds based on amiloride or EIPA and/or an amiloride or EIPA derivative and/or amiloride-like compounds. Specifically, amiloride-like compounds including libraries of such compounds can be computationally tested and/or tested in vitro for their ability to interact with and/or inhibit RdRP biological activity. Further, by identifying RdRP amiloride resistant mutants, the interacting sites of RdRP have been elucidated further facilitating the design of anti-RdRP compounds that interact with interacting sites by various strategies including homology modelling strategies known to those of skill in the art, such as, for example, those described herein.

Reference to the terms “inhibit” or “inhibition” of RdRP activity includes completely or partially and directly or indirectly, inhibiting or reducing or down modulating all or part of one or more activities of one or more RdRPs selected from the picornavirus family.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. compounds are unsuitable active agents for oral compositions or toxic. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs (Wells, Methods Enzymol. 202: 2699-2705, 1991). In this technique, an amino acid residue is replaced by Ala and its effect on the peptide's activity is determined. Each of the amino acid residues of the peptide is analyzed in this manner to determine the important regions of the peptide. In the case of any chemical compound this can be done by sequentially selecting substituents that affect the binding interaction by alterations in, for example, electro donor or acceptor capacity, charge or steric effects in order to identify an optimum scaffold. These parts or residues constituting the active region of the compound are known as its “pharmacophore”. Methods of developing pharmacophores are known in the art (see for example Langer et al. (Eds), Pharmacophores and pharmacophore searches, John Wiley & Sons, Inc, NY, 2006; Reddy et al. (Eds), Free energy calculations in rational drug design, Springer-Verlag, 2001; Martin et al. (Eds), Designing bioactive molecules: Three-dimensional techniques and applications, American Chemical Society, NY, 1998; Wermuth (Ed), The practice of medicinal chemistry, 2^{nd} Edition, Academic Press, NY, 2003, Guner (Ed), Pharmacophore perception, development, and use in drug design, International University Line, 2000 and International Publication No. WO 2003/042702).

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a preferred approach, the atomic coordinates of the three-dimensional structure of target molecules are used for rational drug design. This can be especially useful where the test compound and/or the target RdRP change conformation on binding or form higher order complexes allowing the model to take account of this in the design of the mimetic. Modeling can be used to generate modulators (activators and inhibitors) which interact with the linear sequence or a three-dimensional configuration.

A template molecule is generally selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property of inhibiting RdRP activity or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (BioTechnology 9: 19-21, 1991). In one approach, one first determines the three-dimensional structure of a protein of interest by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., Science 249: 527-533, 1990).

The present invention contemplates, therefore, methods of screening for agents which modulate the activity of RdRP. The methods include assaying for the presence of a complex between the test compound and the RdRP or modulation in the activity of RdRP in the presence of the test compound. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested. In a preferred embodiment, RdRP activity is measured by assessing the amount of RNA produced by the enzyme using radioactive or other detectably modified nucleotides. In a preferred aspect, RdRP activity is measured based on the detection of free pyrophosphate (PPi) which is a product of polymerase mediated nucleotide incorporation into RNA (see in particular Lahser et al Analytical Biochemistry 325:247-245, 2004 which review other suitable methods and is incorporated herein in its entirety by reference). Assays are conveniently suitable for high throughput screening of potential inhibitors.

Another technique for drug screening provides high throughput screening for compounds having suitable binding affinity to a target and is described in detail in Geysen (International Patent Publication No. WO 84/03564). Briefly stated, large numbers of different small test compounds are synthesized on a solid substrate, such as plastic pins or some other surface. The test compounds are reacted with a target and washed. Bound target molecule is then detected by methods well known in the art. This aspect extends to combinatorial approaches to screening for target antagonists or agonists. Purified target can be coated directly onto plates for use in the aforementioned drug screening techniques. However, non-neutralizing antibodies to the target may also be used to immobilize the target on the solid phase.

The present invention also contemplates the use of competitive drug screening assay. In some embodiments amiloride-like compounds capable of specifically binding the RdRP target compete with a test compound for binding to the target or fragments thereof. In this manner, the competitor can be used to detect the presence of any test compound which shares one or more binding sites of the target. The competitors may also be used to discriminate between various higher order forms of a complex comprising a test compound-RdRP complex.

In this embodiment a method for identifying compounds which inhibit RdRP is provided comprising the steps of i) contacting an RdRP or a variant thereof with a competitor amiloride-like compound comprising a detectable label so as to form a complex between the RdRP and the amiloride like competitor compound; ii) measuring a signal from said complex to establish a baseline level; iii) incubating the product of step i) with a test compound; iv) measuring the signal from said complex; and v) comparing the signal from step iv) with the signal from step ii); whereby a decrease in said signal is indicative that said test compound is an inhibitor of RdRP.

In some embodiments, antibodies capable of specifically binding the target compete with a test compound for binding to the target or fragments thereof. In this manner, the antibodies can be used to detect the presence of any test compound which shares one or more antigenic determinants of the target. The antibodies may also be used to discriminate between various higher order forms of a complex comprising a test compound-RdRP complex.

Polypeptide variants are polypeptides having at least 60% amino acid sequence identity with at least one functional domain of a RdRP. Preferably the percentage identity at least 66 or 70% and most preferably 80 or 90 or 95%. A 95% or above identity is most particularly preferred such as 95%, 96%, 97%, 98%, 99% or 100% of all or part of the RdRP Parts include domains or motifs of an RdRP as described herein. Variants also include species of homologs that show at least 60% amino acid identity or more as set out above.

“Percentage similarity or identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

The percent identity or similarity between a particular sequence and a reference sequence such as SEQ ID NO: 1 or SEQ ID NO: 2 is at least about 60% or at least about 70% or at least about 80% or at least about 90% or at least about 95% or above such as at least about 96%, 97%, 98%, 99% or greater. Percentage similarities or identities between 60% and 100% are contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. Preferred identities are at least 60%. Similarity language includes conservative amino acid substitutions and thus is a useful term of art.

The term “molecular replacement”, as used herein, means a method of solving crystal structure using the atomic coordinates of a structurally related molecule. The RdRPs and RdRP variants of the present invention includes all biologically active naturally occurring forms of viral RdRPs as well as biologically active portions, derivatives and variants. Biological activity as used herein refers to the polymerase activity of the polypeptide. Biologically active portions of RdRP include parts of the amino acid sequence of a viral RdRP including without limitation those set out in SEQ ID NO: 1 (amino acid sequence of Poliovirus RdRP) or SEQ ID NO: 2 (amino acid sequence of coxsackievirus) RdRP or any of the sequences described in FIG. 12 or any other publicly available database, or corrected versions thereof. A biologically active portion of a RdRP can be a polypeptide which is, for example, 20, 21, 22, 23, 24, 25, 30, 40, 50, 60, 70, 80, 90, 100, 120, 150, 300, 400 or more amino acid residues in length. Suitably, the portion is a “biologically-active portion” having no less than about 50%, 60%, 70%, 80%, 90%, 99% of the activity of the full-length RdRP polypeptide from which it is derived. Suitable biologically active portions include forms of the polypeptide without all or part of functional domain (interacting site (surface)) or a mutant from. Variant polypeptides includes polypeptides which include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess the desired biological activity of the native protein (e.g., polymerase activity). Such variants may result from, for example, genetic polymorphism or from human manipulation. Biologically active variants of a native RdRP polypeptide will have at least 40%, 50%, 60%, 70%, generally at least 75%, 80%, 85%, preferably about 90% to 95% or more, and more preferably about 98% or more sequence similarity with the amino acid sequence for the native protein as determined by sequence alignment programs described elsewhere herein using default parameters. A biologically active variant of an RdRP polypeptide may differ from that polypeptide generally by as much 100, 50 or 20 amino acid residues or suitably by as few as 1-15 amino acid residues, as few as 1-10, such as 6-10, as few as 5, as few as 4, 3, 2, or even 1 amino acid residue. An RdRP may be altered in various ways including amino acid substitutions, deletions, truncations, and insertions. Methods for such manipulations are generally known in the art. Guidance as to appropriate amino acid substitutions that do not affect biological activity of the protein of interest may be found in the model of Dayhoff et al., (1978) Atlas of Protein Sequence and Structure. Natl. Biomed. Res. Found., Washington, D.C. Conservative substitutions, such as exchanging one amino acid with another having similar properties, may be desirable as discussed in more detail below.

Variant RdRPs may contain conservative amino acid substitutions at various locations along their sequence, as compared to the parent RdRP amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., 1992, Science, 256(5062):1443-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to this scheme is presented in the Table 2.

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of particular amino acids will not have a major effect on the properties of the resulting variant RDRP and that replacement with other amino acids will have a profound effect on the structure of the molecule. Whether an amino acid change results in a functional RdRP can readily be determined by assaying its activity. Conservative substitutions are shown in Table 3 under the heading of exemplary substitutions. More substitutions are shown under the heading of exemplary substitutions. Amino acid substitutions falling within the scope of the invention, are, in general, accomplished by selecting substitutions that do or do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity in vitro or in silico by themselves or in the presence of a test compound.

Alternatively, similar amino acids for making conservative or non conservative substitutions can be grouped into three categories based on the identity of the side chains.

The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, 3^{rd} edition, Wm. C. Brown Publishers (1993).

Thus, a predicted non-essential amino acid residue in a RdRP is typically replaced with another amino acid residue from the same side chain family. Alternatively, mutations can be introduced randomly along all or part of a RdRP coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined.

Accordingly, the present invention also contemplates variants of the naturally-occurring RdRP sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity to an RdRP sequence as, for example, as set forth in any one of SEQ ID NO: 1 or 2 or as set out in FIG. 12. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of the parent RdRP are contemplated. RdRPs also include polypeptides that are encoded by polynucleotides that hybridise under appropriate stringency conditions as known to those in the art (see for example Sambrook, Molecular Cloning: A Laboratory Manual, 3^{rd} Edition, CSHLP, CSH, NY, 2001) especially medium or high stringency conditions, to RdRP sequences, or the non-coding strand thereof.

In some embodiments, variant polypeptides differ from RdRP sequence by at least one but by less than 50, 40, 30, 20, 15, 10, 8, 6, 5, 4, 3 or 2 amino acid residue(s). In another, variant polypeptides differ from the corresponding sequence in any one of SEQ ID NO: 2 by at least 1% but less than 20%, 15%, 10% or 5% of the residues. (If this comparison requires alignment the sequences should be aligned for maximum similarity. “Looped” out sequences from deletions or insertions, or mismatches, are considered differences.). The differences are, suitably, differences or changes at a non-essential residue or a conservative substitution.

A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. An “essential” amino acid residue is a residue that, when altered from the wild-type sequence of an RdRP of the invention, results in abolition of an activity of the parent molecule such that less than 20% of the wild-type activity is present.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a RdRP as, for example, set forth in any one of SEQ ID NO: 1 or 2, and has the activity of that RdRP.

Computational methods may be used to assess whether a variant RdRP falls within the scope of the invention. For example, U.S. Pat. No. 6,782,323 describes a molecular similarity evaluation method comprising obtaining an upper threshold and a lower threshold from one of a value specific to an atom included in a first molecule, a value specific to an atom included in a second molecule of which correlation with respect to the atom in the first molecule is to be evaluated, or another value obtained from these values. The correlation between the atom in the first molecule and the atom in the second molecule is then calculated using the upper and lower thresholds and the similarity between the first and second molecules evaluated based on the correlation. The Molecular Similarity application of SYBYL (Tripos Inc., USA) and QUANTA (Molecular Simulations Inc., USA) are examples of software that will undertake these analyses.

In some embodiments, high resolution X-ray diffraction data collected from crystals saturated with solvent allows the determination of binding positions for solvent molecule. Small molecules that would bind tightly to those sites can then be designed, synthesized and tested for their inhibitory activities.

In other embodiments the subject methods comprises computational screens of small molecule databases for chemical entities or compounds that can bind in whole, or in part, to an RdRP. This screening method and its utility is well known in the art. For example, such computer modelling techniques were described in International Publication No. WO 97/16177.

Once identified by modelling, the subject inhibitors may then be tested for biological activity. For example, the molecules identified may be introduced via standard screening formats into biological activity assays to determine the inhibitory activity of the compounds, or alternatively, binding assays to determine binding (Guthridge et al, Stem Cells, 16:301, 1998). One particularly preferred assay format is the enzyme-linked immunosorbent assay (ELISA). This and other assay formats are well known in the art and thus are not limitations to the present invention.

It is also possible to isolate a target-specific antibody including an antibody to a particular site or to different lower or higher order forms selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

By “match” is meant that the identified portions interact with the surface residues, for example, via hydrogen bonding or by entropy-reducing van der Waals interactions which promote desolvation of the biologically active compound within the site, in such a way that retention of the biologically active compound within the groove is energetically favoured.

It will be appreciated that it is not necessary that the complementarity between ligands and the site extend over all residues lining the surface in order to stabilise binding of the natural ligand. Accordingly, ligands which bind to some, but not all, of the residues lining the surface are encompassed by the present invention.

In general, the design of a molecule possessing stereochemical complementarity determined for example in fitting operations can be accomplished by means of techniques which optimize, either chemically or geometrically, the “fit” between a molecule and a target receptor. Suitable such techniques are known in the art. (See Sheridan et al., Acc. Chem. Res., 20:322, 1987; Goodford, J. Med. Chem., 27:557, 1984; Beddell, Chem. Soc. Reviews: 279, 1985; Hol, Angew. Chem., 25:767, 1986 and Verlinde, W. G. J. Structure, 2:677, 1994, the respective contents of which are hereby incorporated by reference.)

Thus there are two preferred approaches to designing a molecule according to the present invention, which complements the shape of a target binding site. In the first of these, the geometric approach, the number of internal degrees of freedom, and the corresponding local minima in the molecular conformation space, is reduced by considering only the geometric (hard-sphere) interactions of two rigid bodies, where one body (the active site) contains “pockets” or “grooves” which form binding sites for the second body (the complementing molecule, as ligand). The second approach entails an assessment of the interaction of different chemical groups (“probes”) with the active site at sample positions within and around the site, resulting in an array of energy values from which three-dimensional contour surfaces at selected energy levels can be generated.

The geometric approach is illustrated by Kuntz et al, J. Mol. Biol., 161:269-288, 1982, the contents of which are hereby incorporated by reference, whose algorithm for ligand design is implemented in a commercial software package distributed by the Regents of the University of California and further described in a document, provided by the distributor, entitled “Overview of the DOCK Package, Version 1.0,”, the contents of which are hereby incorporated by reference. Pursuant to the Kuntz algorithm, the shape of the cavity represented by the copper-binding site is defined as a series of overlapping spheres of different radii. One or more extant databases of crystallographic data, such as the Cambridge Structural Database System maintained by Cambridge University (University Chemical Laboratory, Lensfield Road, Cambridge CB2 IEW, U.K) and the Protein Data Bank maintained by Brookhaven National Laboratory (Chemistry Dept. Upton, N.Y. 11973, U.S.A.), is then searched for molecules which approximate the shape thus defined.

Molecules identified in this way, on the basis of geometric parameters, can then be modified to satisfy criteria associated with chemical complementarity, such as hydrogen bonding, ionic interactions and van der Waals interactions.

The chemical-probe approach to ligand design is described, for example, by Goodford supra 1984, the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the sites of interest with different chemical probes, e.g., water, a methyl group, an amine nitrogen, a carboxyl oxygen, and a hydroxyl. Favoured sites for interaction between the active site and each probe are thus determined, and from the resulting three-dimensional pattern of such sites a putative complementary molecule can be generated.

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3 DB Unity (Tripos Associates, St. Louis, Mo.).

Programs suitable for pharmacophore selection and design include: DISCO (Abbott Laboratories, Abbott Park, Ill.), Catalyst (Bio-CAD Corp., Mountain View, Calif.), and ChemDBS-3D (Chemical Design Ltd., Oxford, U.K.).

Databases of chemical structures are available from a number of sources including Cambridge Crystallographic Data Centre (Cambridge, U.K.) and Chemical Abstracts Service (Columbus, Ohio).

De novo design programs include Ludi (Biosym Technologies Inc., San Diego, Calif.), Sybyl (Tripos Associates) and Aladdin (Daylight Chemical Information Systems, Irvine, Calif.).

Those skilled in the art will recognize that the design of a mimetic compound may require slight structural alteration or adjustment of a chemical structure designed or identified using the methods of the invention.

RdRP mutants may also be generated by site-specific incorporation of unnatural amino acids into the human protein using the general biosynthetic method such as Noren et al, Science, 244:182-188, 1989. In this method, the nucleotides encoding the amino acid of interest in wild-type RdRP is replaced by a “blank” nonsense codon, TAG, using oligonucleotide-directed mutagenesis. A suppressor directed against this codon, is then chemically aminoacylated in vitro with the desired unnatural amino acid. The aminoacylated residue is then added to an in vitro translation system to yield a mutant enzyme with the site-specific incorporated unnatural amino acid. Examples of unnatural amino acids are listed in Table 4.

In other aspects, the present invention provides methods of treating or diagnosing subjects. Preferably, the subject is a human. The present invention contemplates, however, primates, livestock animals, laboratory test animals, companion animals and avian species as well as non-mammalian animals such as reptiles and amphibians. The methods therefore have applications, therefore, in human, livestock, veterinary and wild life therapy and diagnosis.

Viruses contemplated in the Picornaviridae family include but are not limited to those listed in Table 6.

Pharmaceutical compositions suitable for use in the present invention are contemplated and are as would be formulated, prepared and administered as appropriately determined by skilled addressee.

Pharmaceutical compositions are conveniently prepared according to conventional pharmaceutical compounding techniques. See, for example, Remington's Pharmaceutical Sciences, 18th Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract. See for example, International Patent Publication No. WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

The actual amount of active agent administered and the rate and time-course of administration will depend on the nature and severity of the picornavirus infection. Prescription of treatment, e.g. decisions on dosage, timing, etc. is within the responsibility of general practitioners or specialists and typically takes account the condition of the individual patient, the site of delivery, the method of administration and other factors known to practitioners. Examples of techniques and protocols can be found in Remington's Pharmaceutical Sciences, supra.

The pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.1 ng, 0.2 ng, 0.3 ng, 0.4 ng, 0.5 ng, 0.6 ng, 0.7 ng, 0.8 ng. 0.9 ng, or 0.1 mg, 0.2 mg, 0.3 mg, 0.4 mg, 0.5 mg, 0.6 mg, 0.7 mg, 0.8 mg. 0.9 mg to about 1 to 10 mg or from 5 to 50 mg of agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The agents may be administered in a convenient manner such as by the oral, intravenous (where water soluble), intraperitoneal, intramuscular, subcutaneous, intradermal or suppository routes or implanting (e.g. using slow release molecules). The agent or composition comprising the agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulfate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

The present invention is further described by the following non-limiting Examples.

### Example 1

### Amiloride and Amiloride Derivatives are Active Against Coxsackievirus

In a first set of experiments, the antiviral potency of a range of structurally related ion channel inhibitors (amiloride derivatives) was assessed.

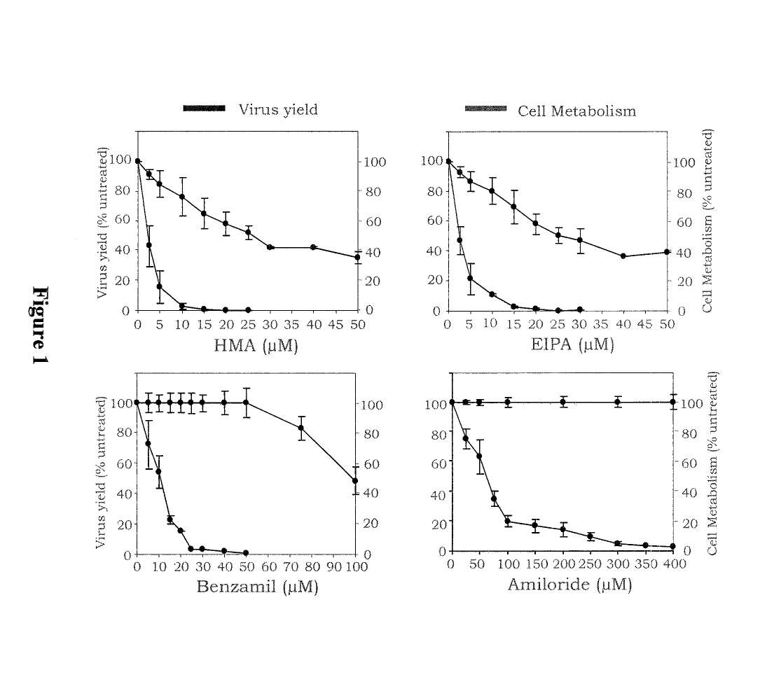

As shown in FIG. 1 the antiviral effect (expressed as % virus yield compared to untreated cultures) and the cytotoxic effect (expressed as % cell metabolism in the Alamar Blue assay compared to untreated cells) for amiloride, EIPA, benzamil and HMA against Coxsackievirus B3 (CVB3) in HeLa cells. Each of the drugs showed a selective antiviral effect against CVB3, as shown previously for rhinoviruses.

### Example 2

### Amiloride and Amiloride Derivatives Inhibit Intra-Cellular Virus Replication

In a second set of experiments, the dependence of antiviral effect on the time of addition of drugs (amiloride and EIPA) was determined.

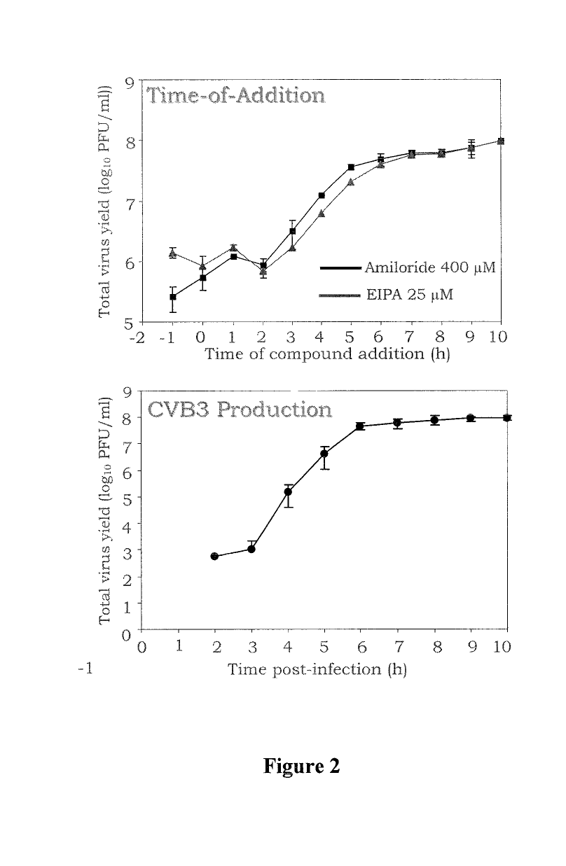

As shown in FIG. 2 the amount of virus produced by cultures which were infected with CVB3 (multiplicity of 1 plaque forming unit per cell), where 400 μM amiloride or 25 μM EIPA was added to the cultures at 1 hour intervals. Cultures were incubated until 10 hours post-infection, then the total virus yield (released virus+intracellular virus) was quantitated (FIG. 2A). Kinetics of virus production was measured in parallel (FIG. 2B). The results demonstrate that amiloride and EIPA are fully effective in reducing virus replication when added to infected cells up to 2 hours post-infection, which indicates that they do not have a significant effect on the binding, entry or uncoating of the virus. This contrasts with well-known antiviral drugs active against picornaviruses such as Pleconaril, which prevent uncoating and/or binding to the cell. In addition, the kinetics by which amiloride and EIPA continue to inhibit replication closely follows the kinetics of virus replication, which suggests that these drugs inhibit an intracellular replication event.

### Example 3

### Amiloride and Amiloride Derivates Affect Virus Replication at the Stage Between Virus Uncoating and Virus Release

In a further set of experiments, the effect of the drugs on the release of virus from infected cells was determined.

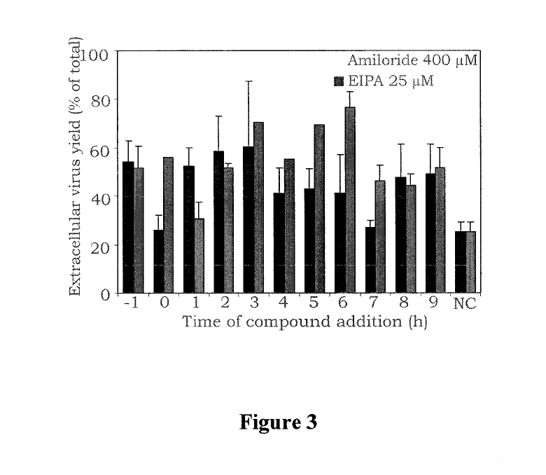

As shown in FIG. 3 the proportion of virus released from cells infected with CVB3 and treated with amiloride or EIPA, added at various times as shown in FIG. 2, compared to untreated controls (NC). Both amiloride and EIPA resulted in a slight increase in the proportion of extracellular virus, in contrast to the reduced level of virus release previously reported for these drugs with Rhinoviruses (Gazina et al., 2005 (supra)). This suggests that the stage of virus replication affected by these drugs is between virus uncoating and virus release.

To assess whether the antiviral effects of amiloride and EIPA were reversible, HeLa cells were infected in the presence of the compounds, which were then removed from the culture medium at various times post-infection. Virus replication was then allowed to continue until 10 hours post-infection. When the compounds were removed at 1, 2 or 4 hours post-infection, CVB3 production at 10 hours post-infection was equivalent to that of the untreated cultures. Exposure to the compounds beyond 4 hours caused progressive reduction in CVB3 yields. This demonstrated that the predominant antiviral effect of EIPA and amiloride on CVB3 is the reversible inhibition of the intracellular virus replication (i.e. RNA replication, protein synthesis/processing or virus assembly). Benzamil also predominantly affected this stage of the CVB3 replication cycle (data not shown), suggesting that the three compounds are likely to have the same mechanism of action.

### Example 4

### Virus Production in the Presence of Amiloride or Amiloride Derivatives is Directly Proportional to the Amount of Viral RNA Produced

In a further set of experiments, the effect of the drugs on viral assembly was determined. Virus-infected cultures were treated with amiloride or EIPA, or with guanidine hydrochloride (GHCl) at 500 micromolar, giving a similar antiviral effect to amiloride and EIPA, or at 2 millimolar giving a still higher antiviral effect. Guanidine is well known to inhibit the initiation of viral RNA replication through its effect on the viral 2C protein, but has no effect on the assembly of virus from RNA that is replicated in its presence. Infected cells were incubated with 3H-uridine to label viral RNA, and RNA was detected by electrophoresis and autoradiography.

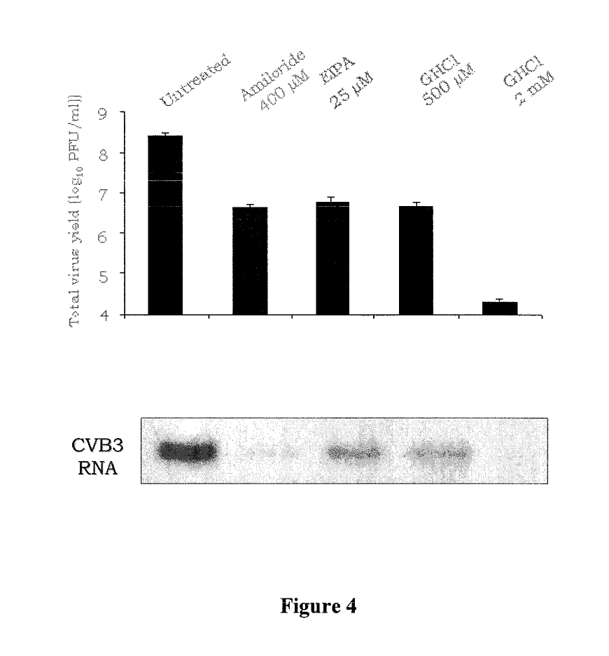

As shown in FIG. 4 the amount of virus produced in cells is directly proportional to the amount of viral RNA produced. Infected cells treated with 25 micromolar EIPA or with 500 micromolar guanidine produced equivalent amounts of viral RNA, and equivalent amounts of virus, suggesting that EIPA does not affect virus assembly. Infected cells treated with 400 micromolar amiloride showed greatly reduced levels of viral RNA by 3H-uridine labelling, despite having similar amounts of virus to cultures treated with EIPA. However, this appears to be due to indirect effects of amiloride on the ability of the cell to take up uridine from the culture medium, and is consistent with amiloride having no effect on virus assembly.

### Example 5

### Amiloride and Amiloride Derivatives have No Effect Upon Translation of Viral RNA into Viral Proteins

Picornavirus RNA replication and protein synthesis are coupled processes, and inhibition of one of them indirectly inhibits the other. A standard method to determine which of the two processes is inhibited directly is to add the inhibitor and pulse-label viral RNA and proteins at the time in the replication cycle when sufficient amounts of RNA and proteins have been produced to allow continuation of one process independent of the other.

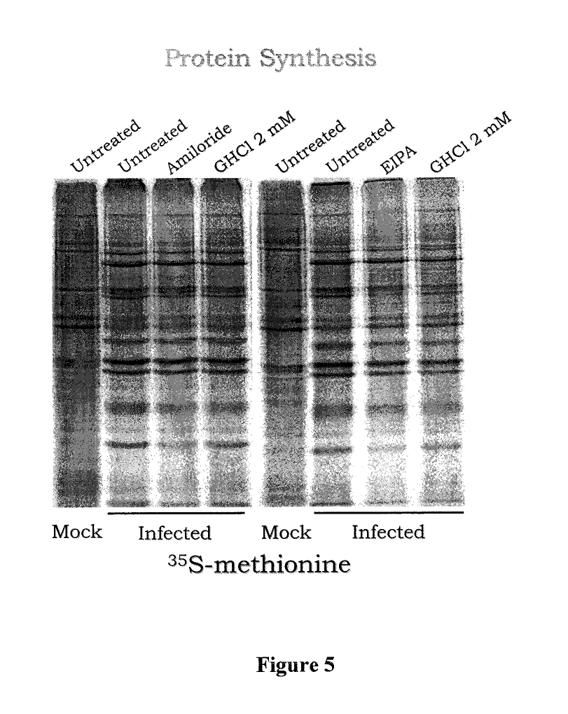

In a further set of experiments, the effect of the drugs on viral protein synthesis (see FIG. 5) and viral RNA synthesis (see FIG. 6) was determined. Cells were infected as described in Examples 2 and 3, and drugs were added to cells at four hours post infection. Radioactive labels (35S-methionine for proteins, 3H-uridine for RNA) were added to cells from 4.5-5 h post infection. Treatment with GHCl, which inhibits enteroviral RNA replication but not protein synthesis was used as a control.

As shown in FIG. 5 the amount of viral protein synthesised in the presence of the drugs is not reduced significantly. This suggests that once the viral, messenger RNA is produced (through the process of RNA replication), the drugs have no effect on translation of the viral RNA into viral proteins.

### Example 6

### Amiloride Targets Viral RNA Replication

FIG. 6 shows that the amount of viral RNA synthesised in the presence of the drugs is significantly reduced. FIG. 6A shows the kinetics of viral RNA replication, indicating that the peak of viral RNA replication under these conditions occurs between four and five hours post infection. FIG. 6B shows that when infected cells are treated with amiloride or EIPA during this time, the amount of viral RNA synthesis is dramatically reduced compared to untreated cells. This is similar to the reduction observed in guanidine treated cells, where guanidine is known to directly inhibit viral RNA replication. These results suggest that amiloride and EIPA have a direct effect on viral RNA replication.

While these results demonstrate that amiloride and EIPA have a direct effect on viral RNA replication, they do not distinguish between effects on the assembly of replication complexes (including initiation of viral RNA replication), which is the mechanism of action of guanidine by its effect on the viral 2C protein, and effects on viral RNA replication per se, for example by inhibition of the enzymatic activity of the viral RNA-dependent RNA polymerase.

### Example 7

### Amiloride Targets RNA Dependent RNA Polymerase

To determine the mechanism of action, CVB3 was passaged sequentially in the presence of amiloride in six separate cultures. Virus that had been passaged in the presence of drug was plaque purified and examined for resistance to amiloride. HeLa cells infected with the passaged (putative drug-resistant) viruses or with wild-type virus (passaged in the absence of drug) were examined for the production of virus in the presence of amiloride. This is described in more detail below.

The results in FIGS. 1-6 suggested that amiloride and its derivatives may inhibit a viral protein involved in CVB3 RNA replication. To test this hypothesis, HeLa cells were transfected with CVB3 (Nancy) RNA produced from the p53CB3/T7 plasmid (van Ooij et al 2006; Wessels et al 2005), and the resulting virus was passaged in the presence of 400 μM amiloride or without treatment. Amiloride rather than EIPA was chosen for passaging due to its low toxicity. After 13 passages, virus yields in amiloride treated cultures became similar to those in untreated. At that stage, 6 isolates of amiloride passaged virus as well as two isolates of untreated virus were plaque purified. To confirm amiloride-resistance of amiloride-selected isolates, HeLa cells were infected with the 6 viruses at an MOI of 0.01 and incubated with 400 μM amiloride 5 for 48 hours, or left untreated. Virus yields in the samples were then measured by plaque assay. The results showed that all isolates of amiloride-selected viruses had similar levels of resistance to amiloride, with virus yields in the presence of the compound being on average 45% of the yields in its absence.

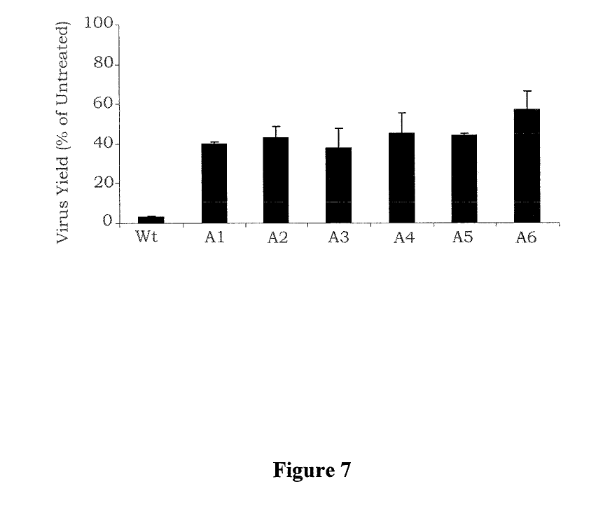

As shown in FIG. 7 all six clones passaged in the presence of amiloride show reduced inhibition of virus replication in the presence of drug, compared to wild-type virus. These results demonstrate that mutation(s) in viral proteins are sufficient to overcome the antiviral effects of amiloride, suggesting that the drug may be acting directly on a viral protein rather than on a cellular protein that is recruited by the virus. However, it should be noted that picornaviruses are also able to develop resistance to some drugs such as brefeldin A, which act on cellular proteins, by the acquisition of mutations that allow the virus to use redundant biochemical pathways in the cell rather than the brefeldin-sensitive pathway.

### Example 8

### Amiloride and Guanine have Different Mechanisms of Action

Amiloride and EIPA are acylguanidine compounds, and share the guanidine group that is well known to inhibit RNA replication in most Picornaviruses.

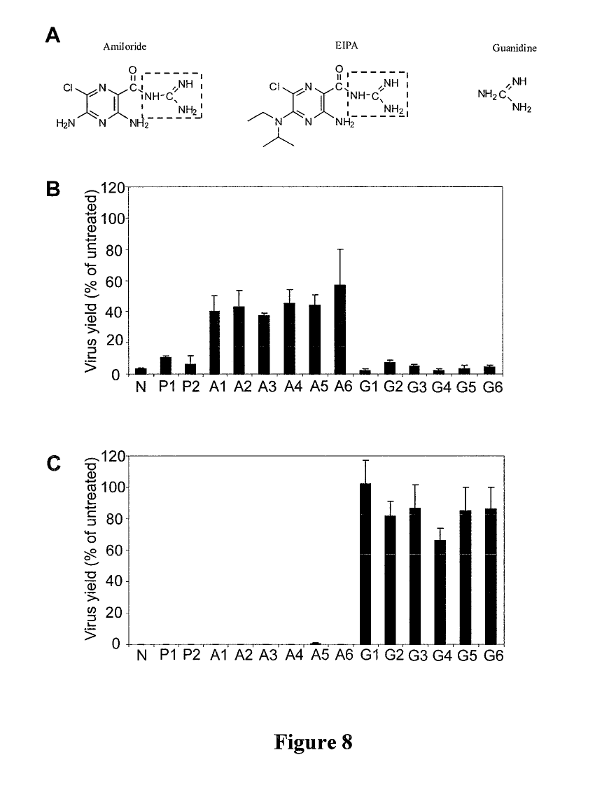

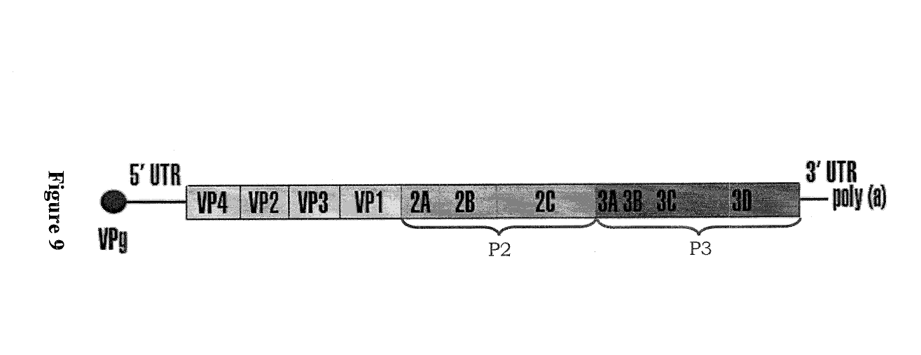

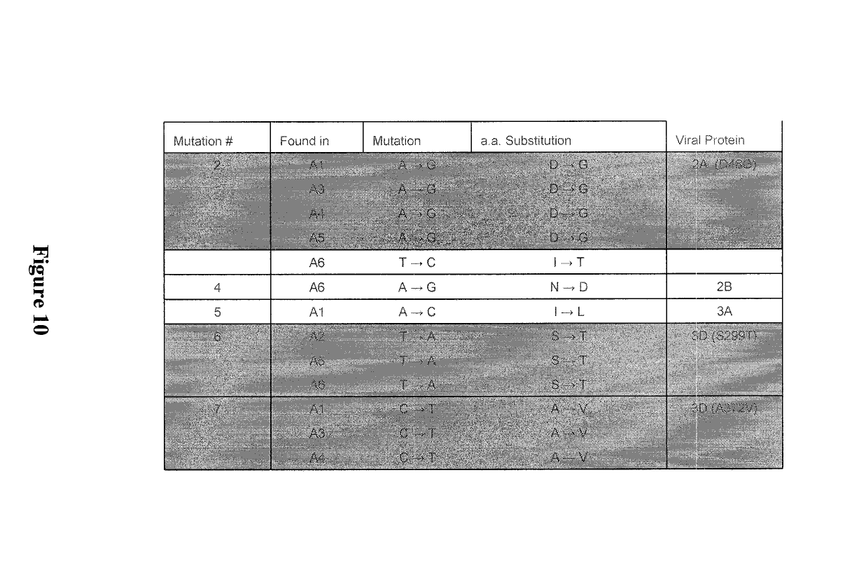

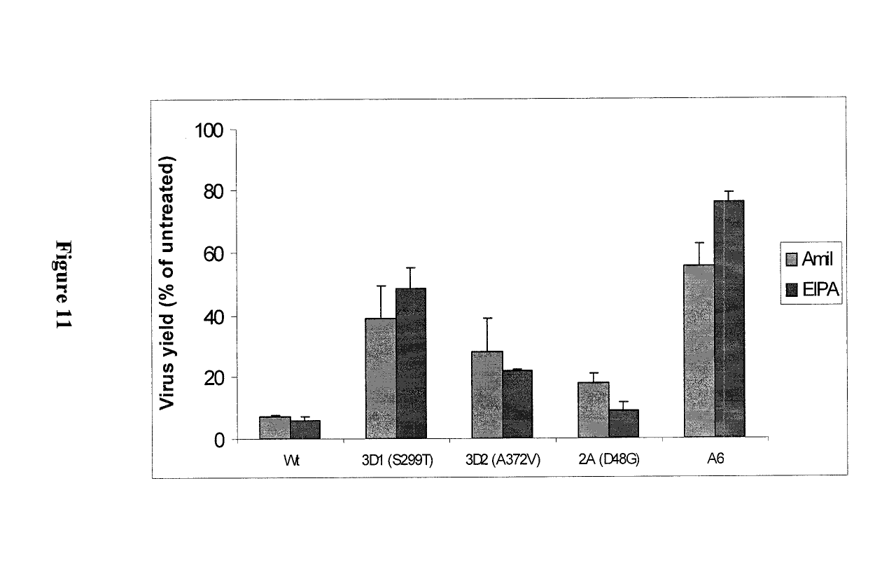

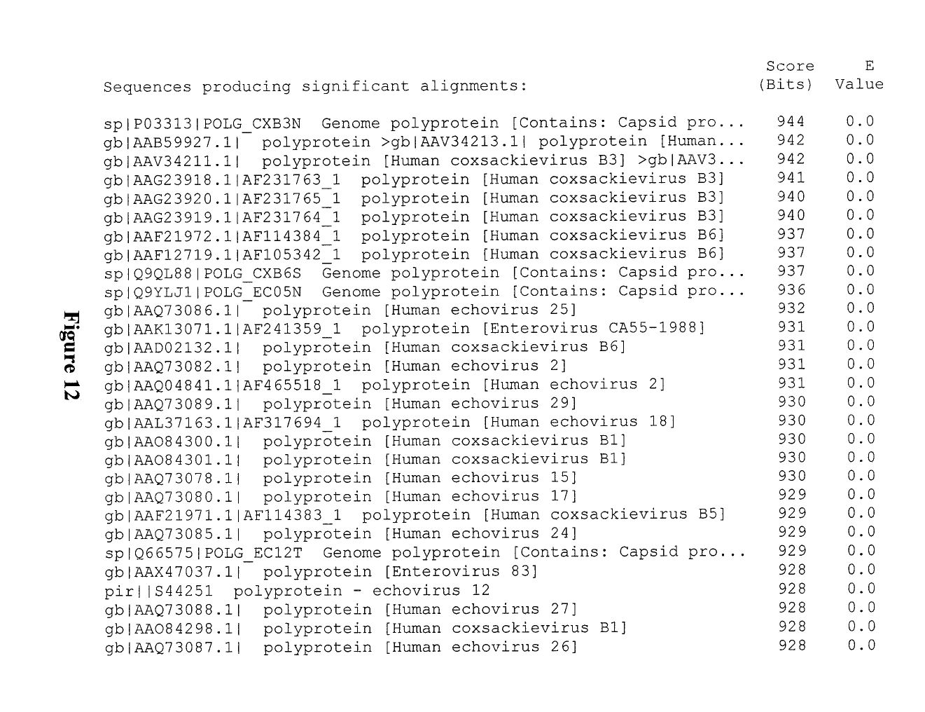

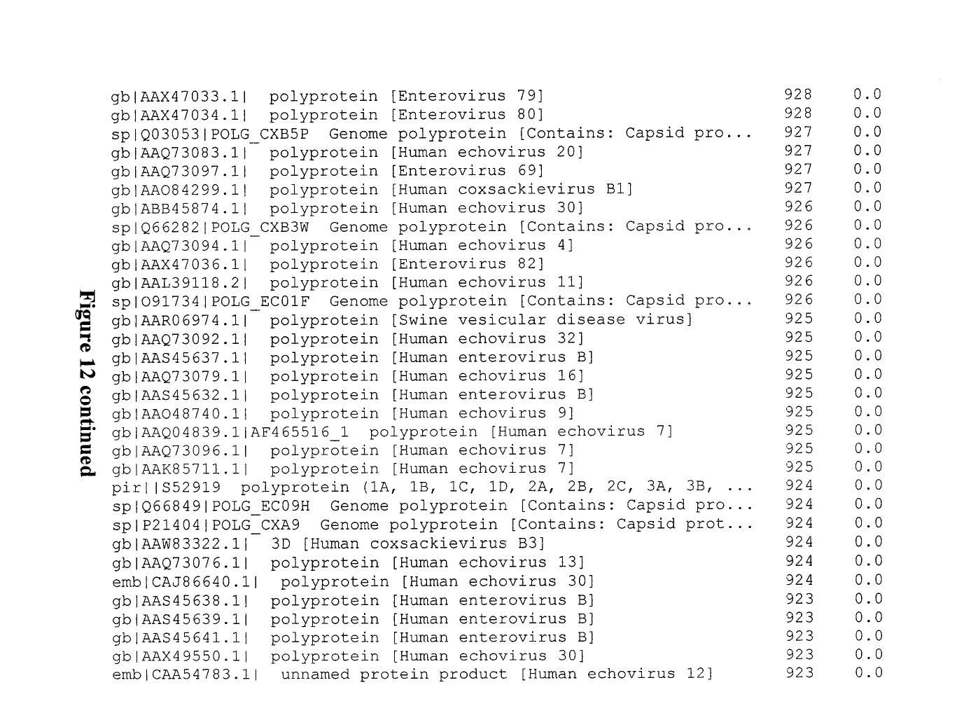

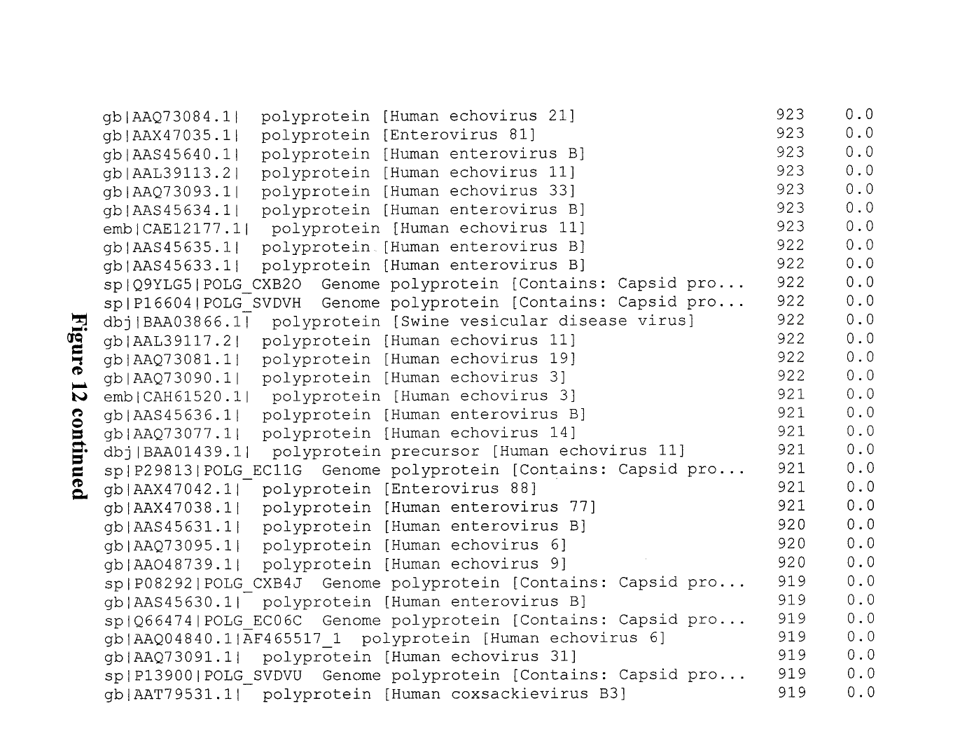

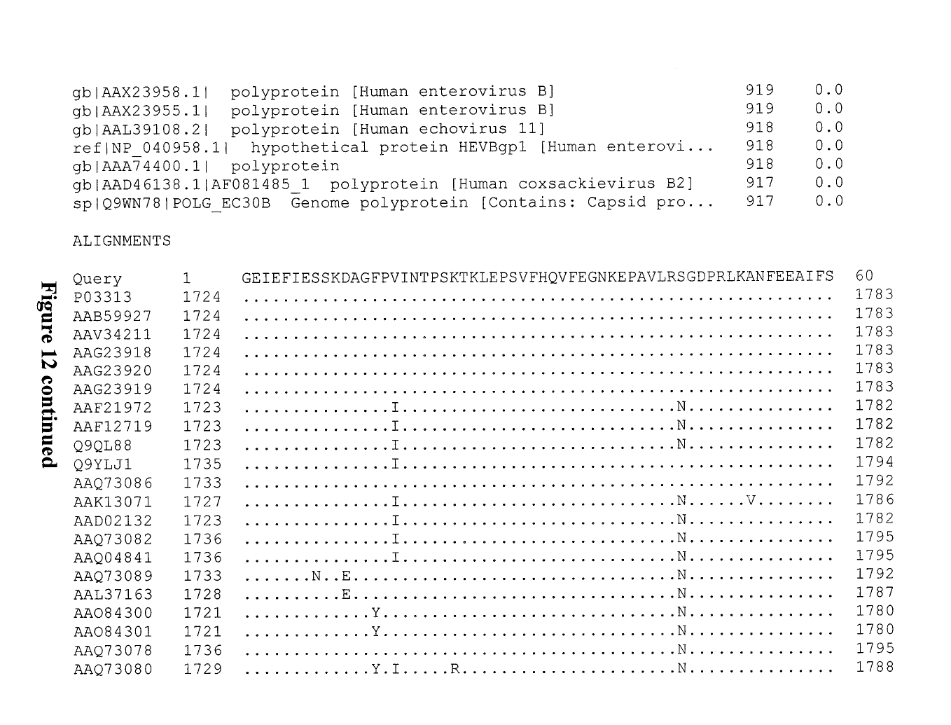

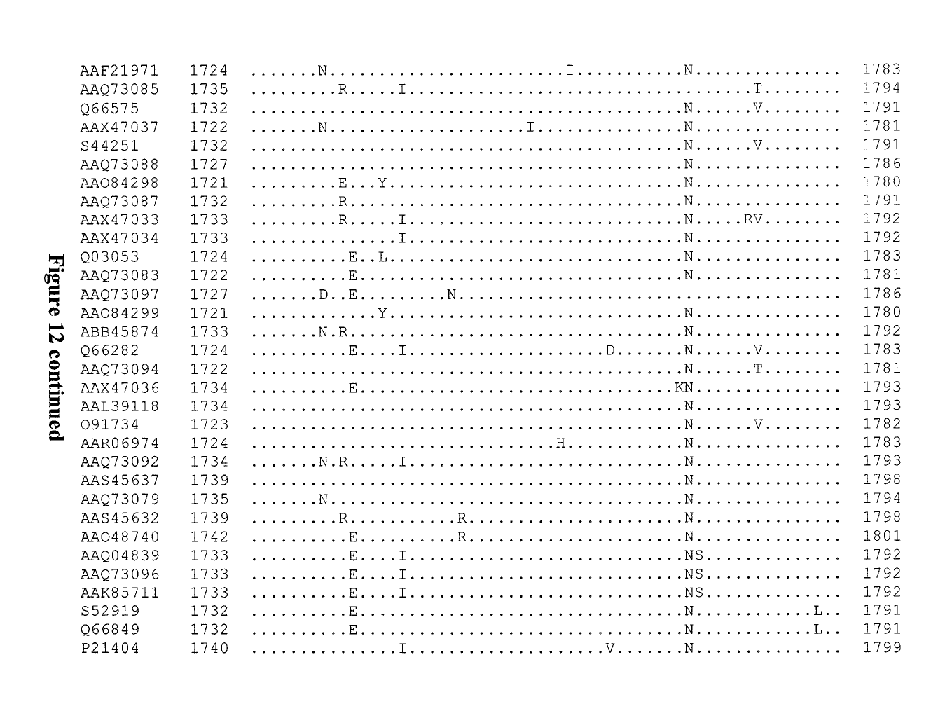

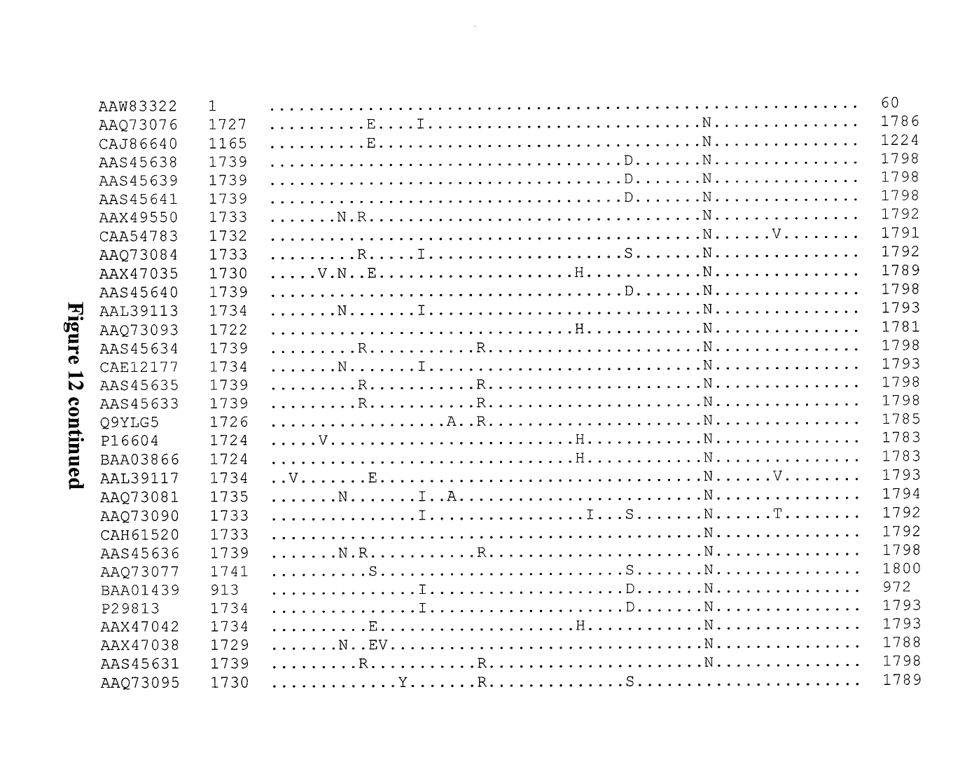

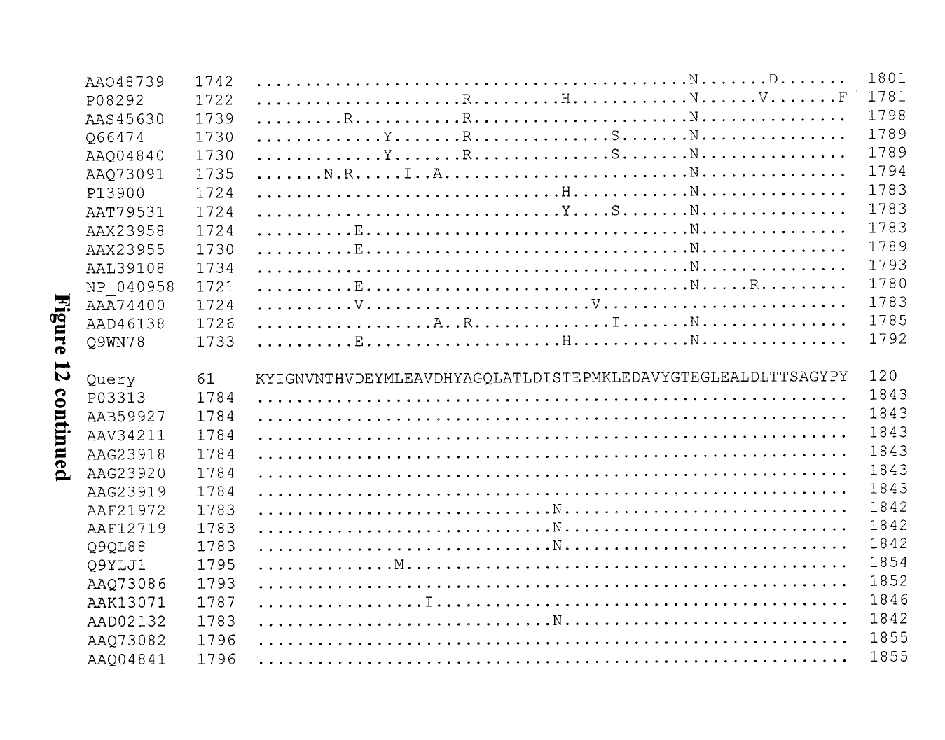

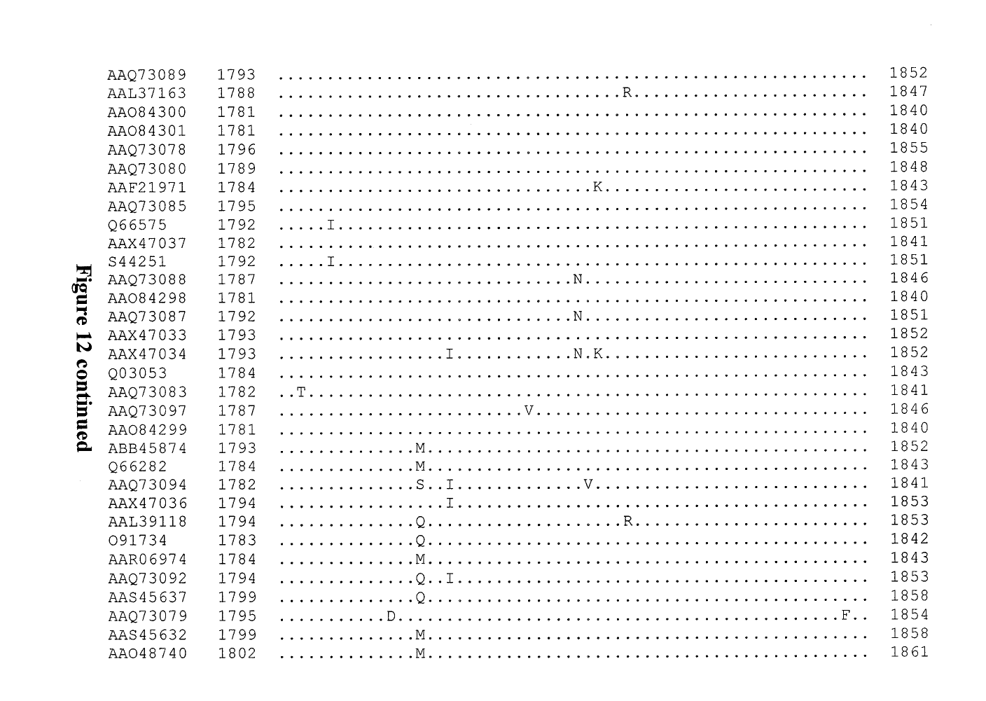

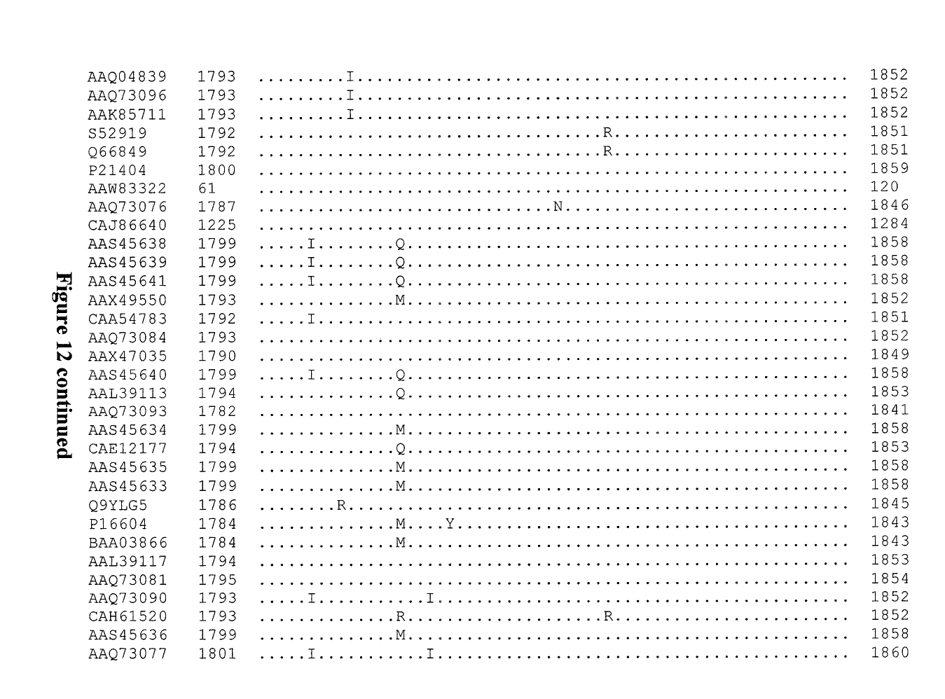

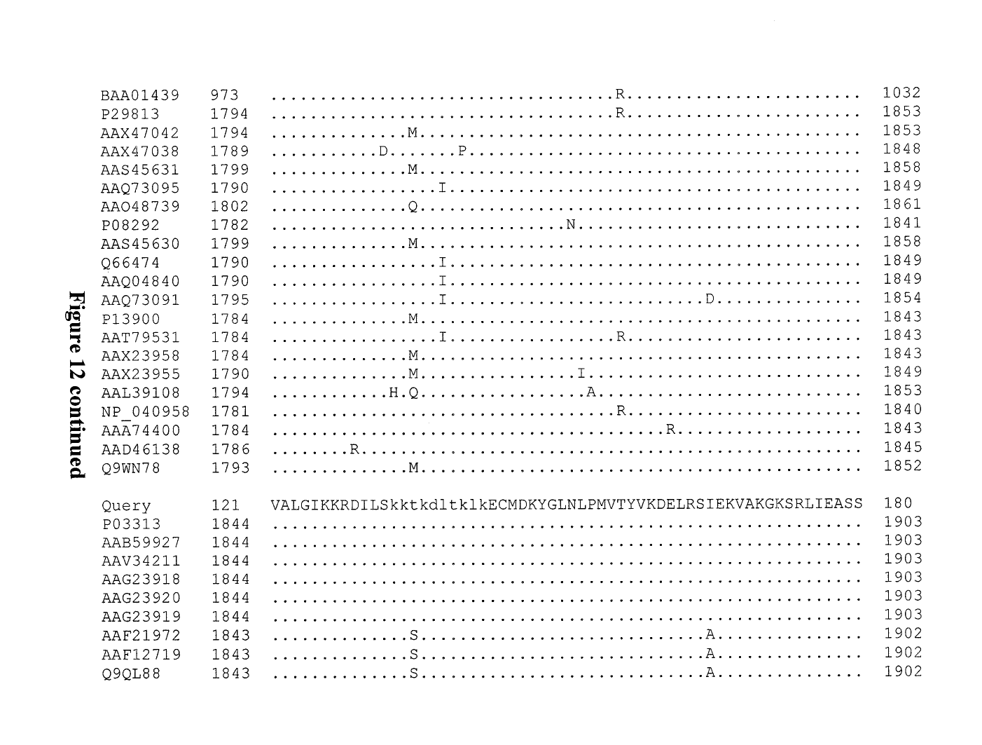

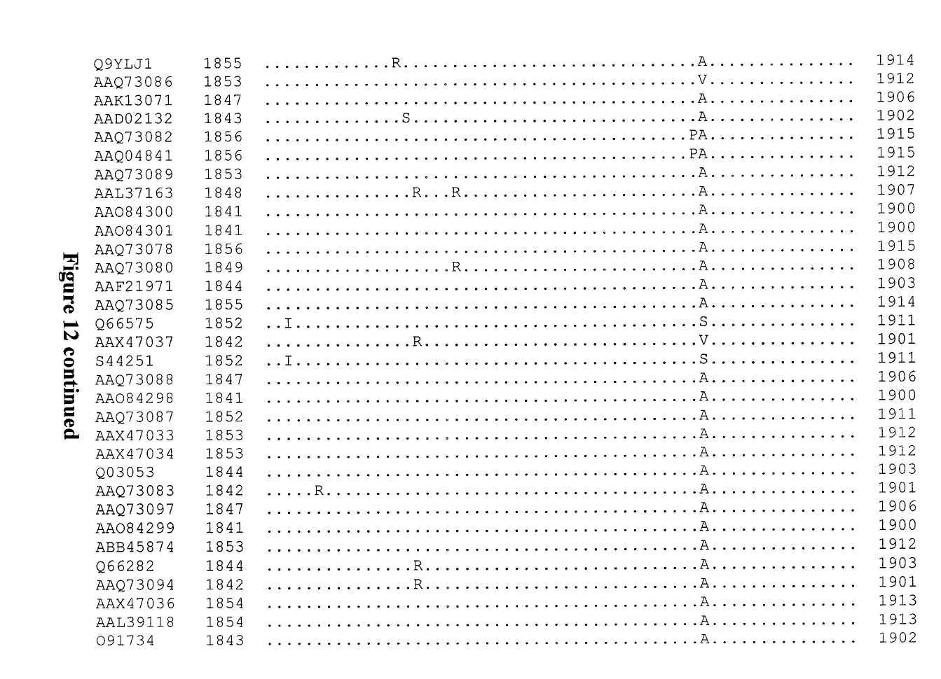

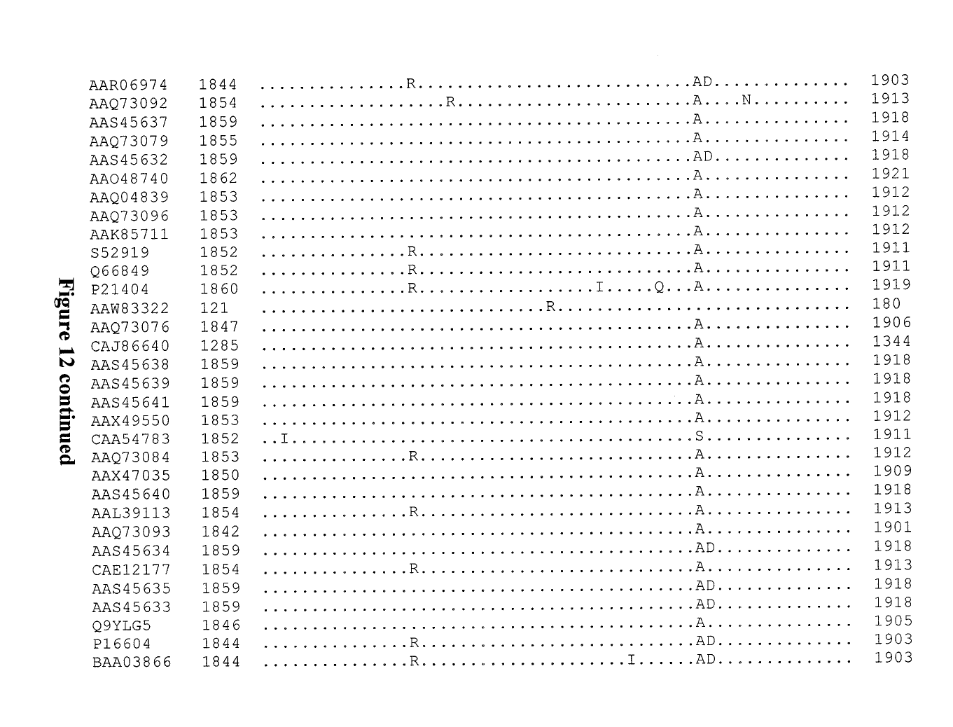

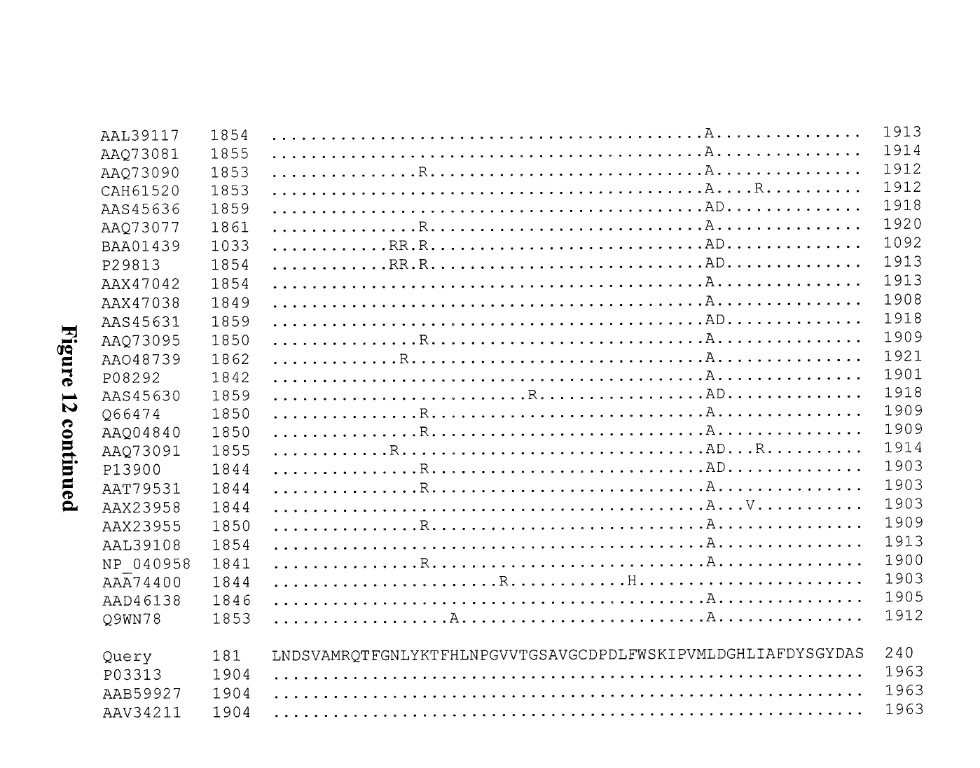

FIG. 8 shows that the six clones of amiloride-resistant virus remained sensitive to the antiviral effects of guanidine, and that six clones of guanidine-resistant virus (prepared by sequential passaging in the presence of guanidine) remained sensitive to the antiviral effects of amiloride. These results indicate that amiloride and guanidine have different mechanisms of antiviral action, despite sharing the structural similarity of the guanidine group.

### Example 9

### Amiloride Resistant RdRp Mutants have Mutations in the E Motif or NTP Binding Site of RdRP

To gain further insight into the mechanism of antiviral action of amiloride (and amiloride derivatives), the P2 and P3 regions of the genome of each of the six amiloride-resistant clones was sequenced, and the encoded protein sequence was compared with the known wild-type sequence for CVB3 (SEQ ID NO: 2)

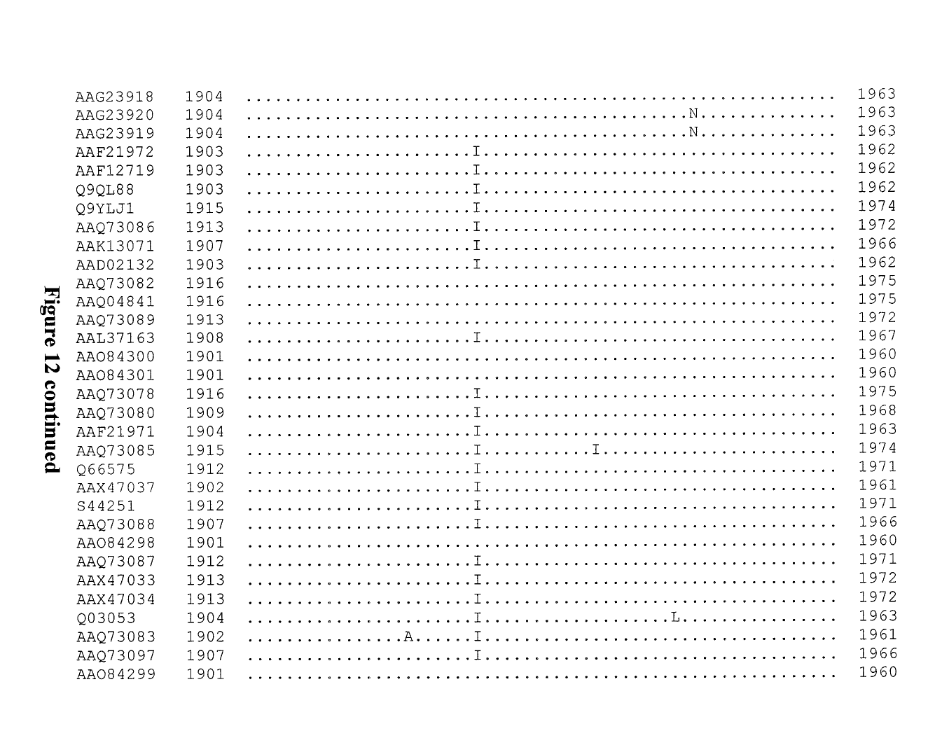

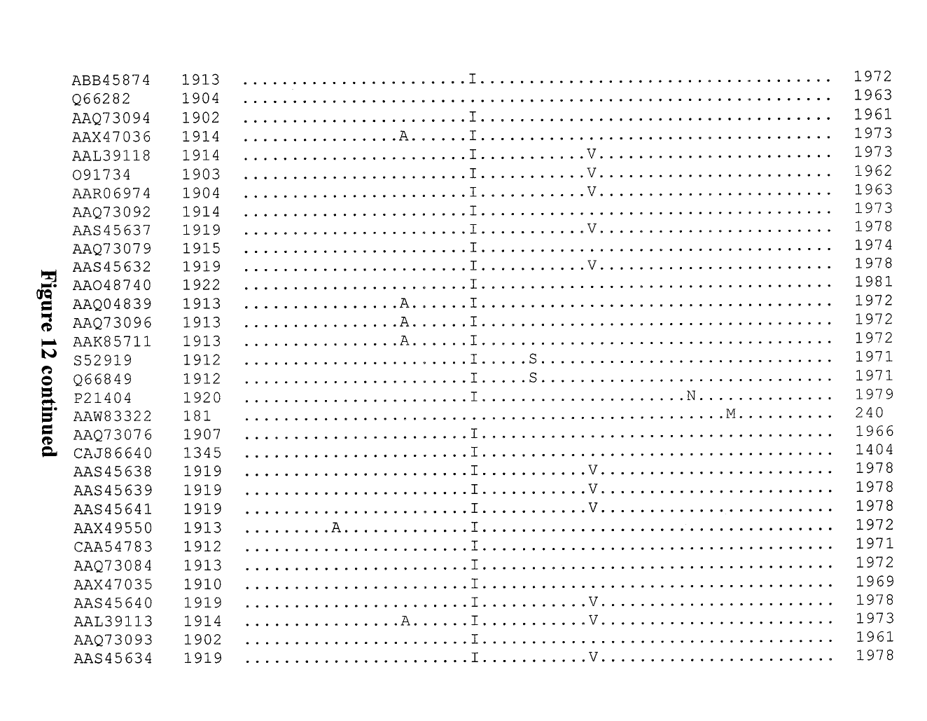

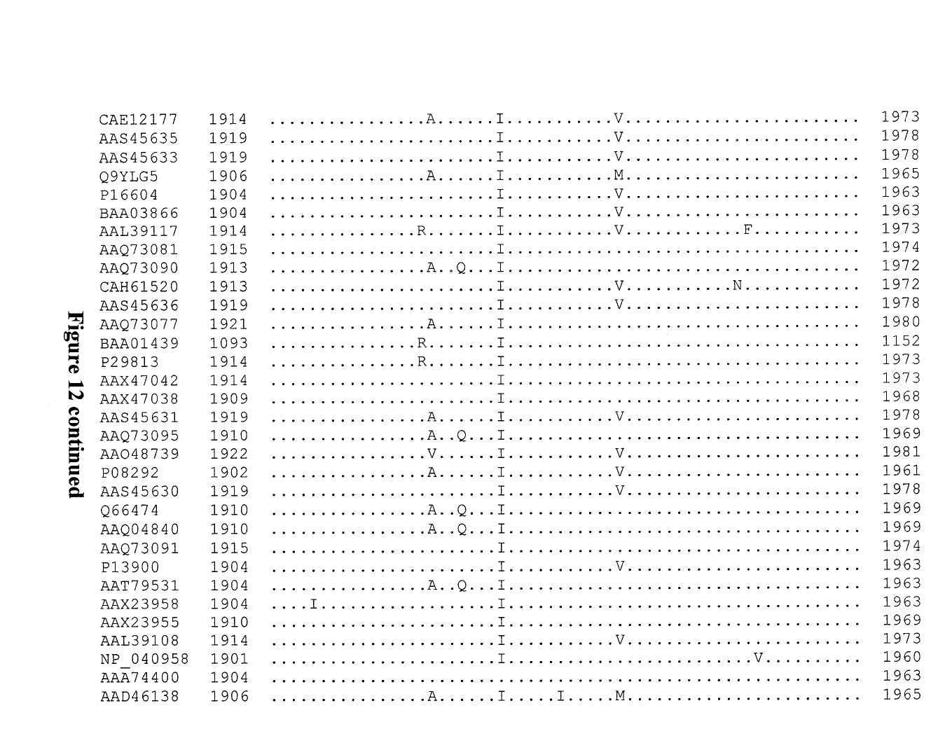

FIG. 9 shows a schematic representation of the genome and encoded proteins of CVB3. CVB3 encodes a single polyproteins which is then cleaved by viral proteases to yield at least eleven different viral proteins which are nominally assigned to three regions. The P1 region contains the viral capsid structural proteins, VP1, VP2, VP3 and VP4; because amiloride did not affect viral attachment, uncoating or release it was considered unlikely that drug-resistance mutations would be found in this region, and it was not sequenced. The P2 region contains non-structural proteins involved in viral replication, 2A, 2B and 2C. 2B is a membrane-spanning protein but does not have any other resemblance to known ion channel proteins. 2C is a membrane-spanning protein that functions to associate the 3AB protein with the membranous replication complex, and is the target for inhibition of RNA replication by guanidine. The P3 region contains further non-structural proteins involved in RNA replication; the 3A protein, the 3B protein (VPg) which acts as a primer for RNA replication; the 3C protein that is the virus-encoded protease, and the 3D protein that is the viral RNA-dependent RNA polymerase. The P2 and P3 regions of the genome were sequenced.

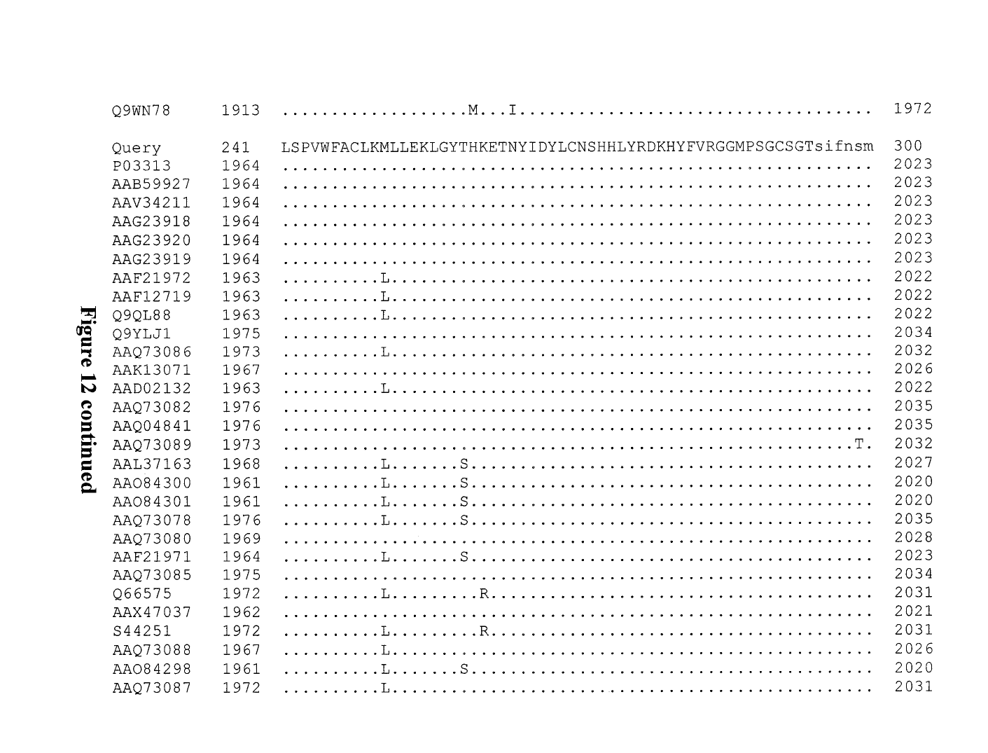

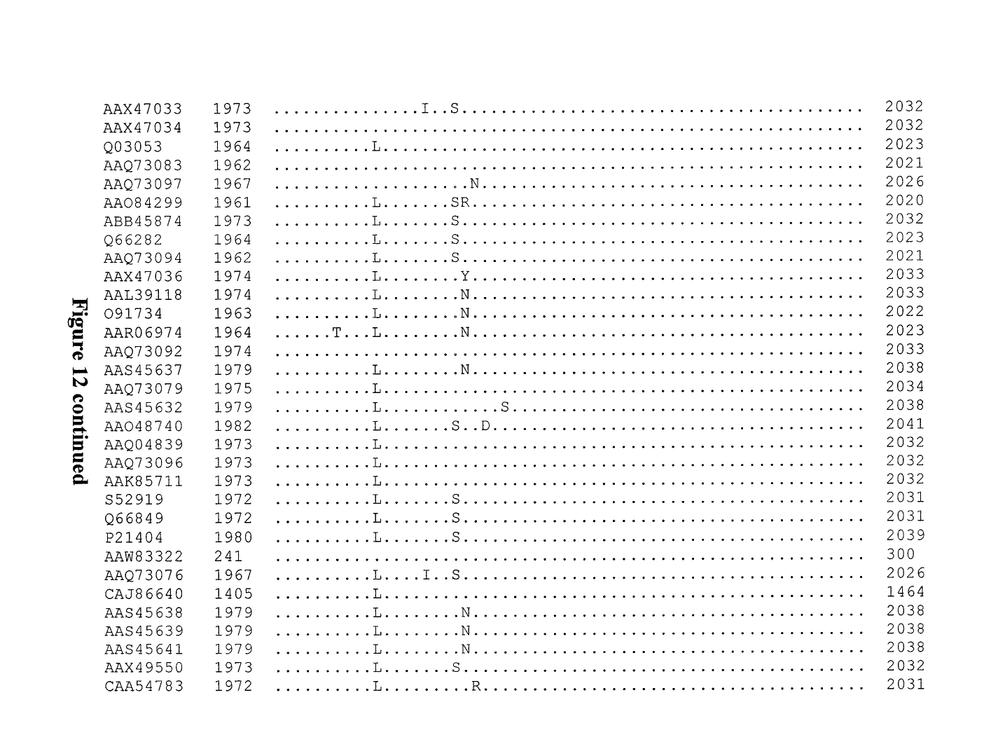

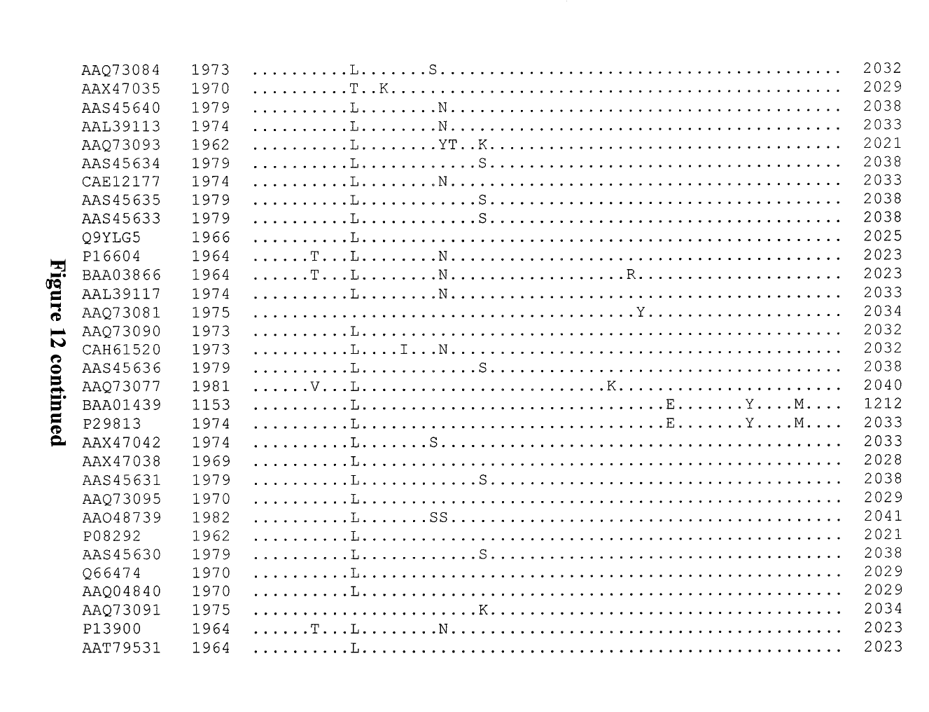

FIG. 10 shows the nucleotide and deduced amino acid sequences of the mutations found in the 6 amiloride-resistant clones. All isolates had a mutation within the 3D protein (viral RNA dependent RNA polymerase): S299T in three isolates, and A372V in the other three isolates. Four of six isolates had the D48G mutation in the 2A protein. Single and three letter abbreviations for amino acid residues used in the specification are defined in Table 5.

To determine the precise target for the antiviral action of amiloride, each of the 3 mutations shown in FIG. 10 were separately introduced into a full-length, infectious clone of CVB3 and the effect of drugs on the progeny, mutant virus was determined.

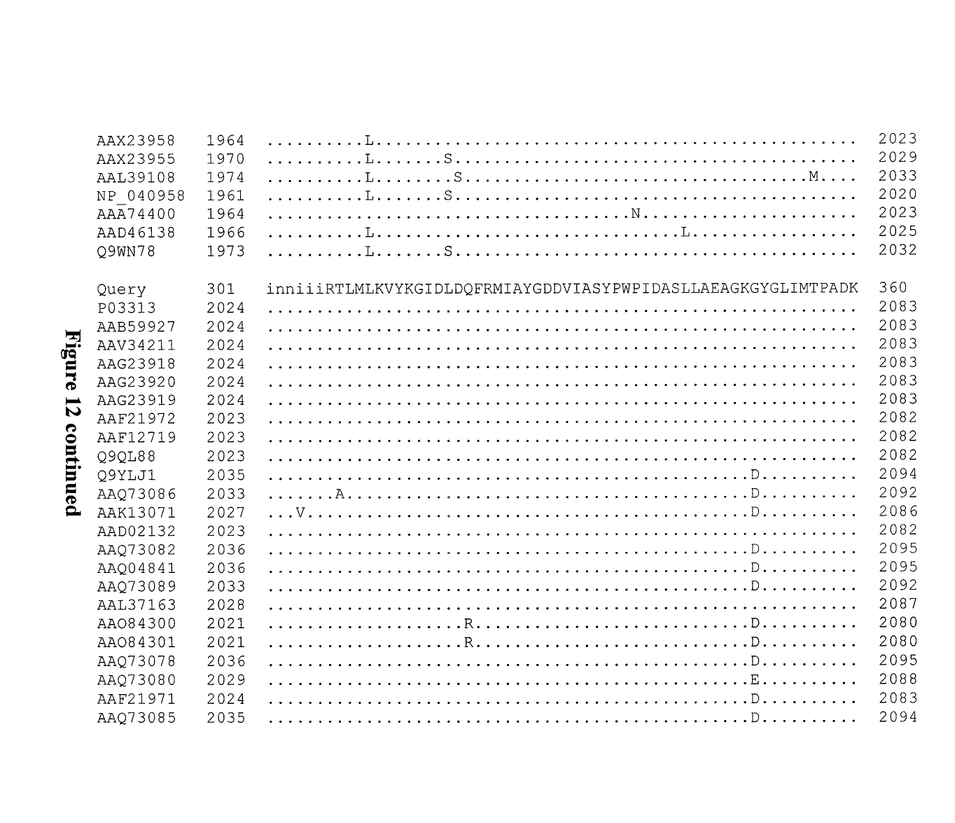

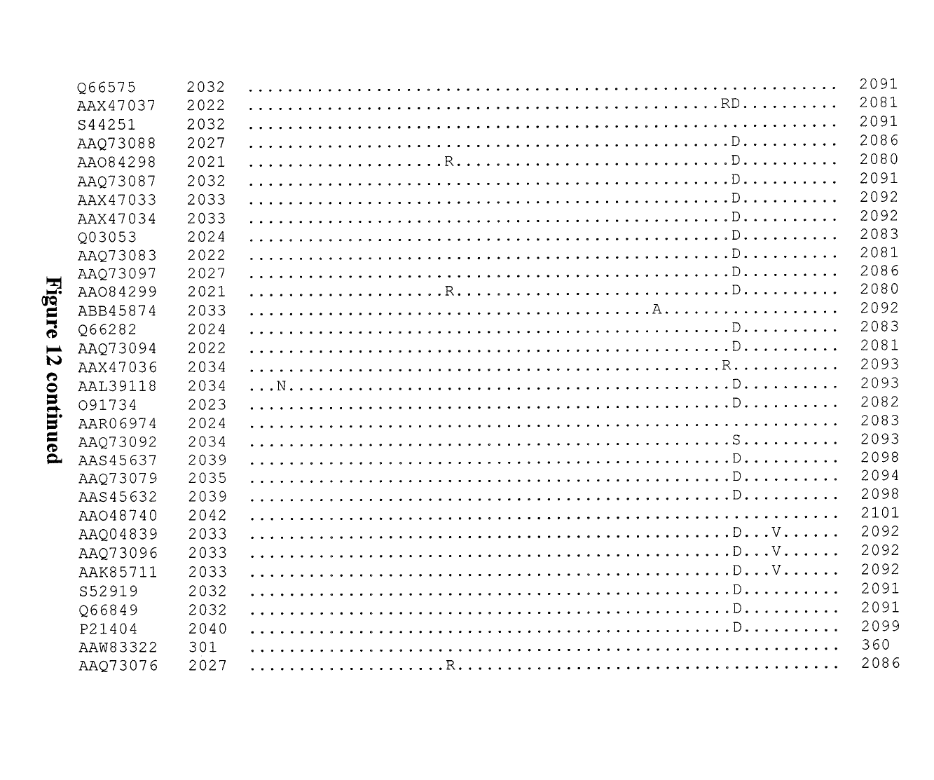

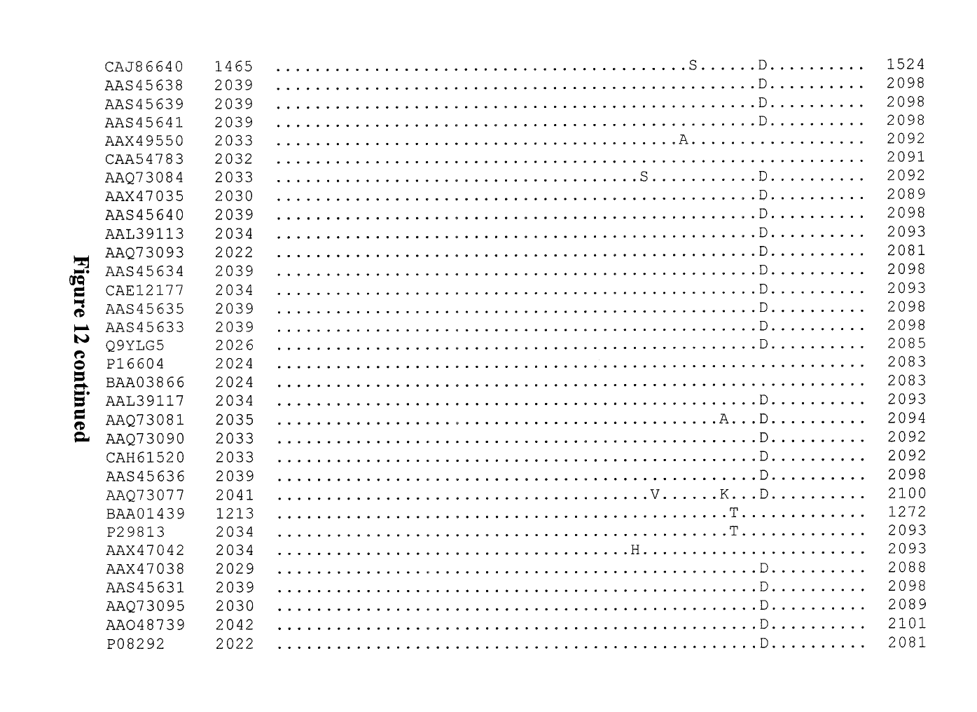

As shown in FIG. 11 viruses containing either S299T or A372V mutations show reduced sensitivity to both amiloride and EIPA, demonstrating that these amino acids are important in the mechanism of action of the drugs. In contrast, the D48G mutation, by itself, had no significant effect on the virus sensitivity to the drugs. It is possible that the D48G mutation may have activity when combined with S299T and/or A372V mutations, which could be tested using the scheme described for the individual mutations.

The S299T mutation is very close to the nucleotide triphosphate (NTP)-binding centre of CVB3 polymerase. The A372V mutation is in the E motif of the polymerase which is part of the active centre, helping to position the 3′ end of the primer strand during RNA elongation Therefore, in some embodiments, amilorides inhibit the enzymatic activity of the polymerase by binding in its active centre.

Amiloride-like compounds such as amiloride and its derivatives, EIPA and benzamil, inhibit reproduction of HRV2 in HeLa cells and the antiviral activity of these compounds was unlikely to be due to inhibition of their normal cellular targets. As demonstrated herein, these compounds have a stronger antiviral effect against CVB3 than against HRV2 but with the same order of antiviral potency: EIPA>benzamil>amiloride. Despite this apparent similarity, the mechanisms of antiviral activity are significantly different between the two picornaviruses.

The antiviral activity of amiloride and EIPA against CVB3 is due to the inhibition of RNA replication, while no effect of the compounds upon HRV2 RNA replication has been observed. Additionally, previous data have shown an inhibitory effect of EIPA on HRV2 release whereas the release of CVB3 was not inhibited by EIPA or amiloride. Amiloride-resistant CVB3 isolates had either a S299T mutation in 3Dpol or a combination of two mutations: A372V in 3Dpol and D48G in the 2A protein (one isolate with the S299T mutation also had the D48G mutation). Both 3Dpol mutations, when individually introduced into the CVB3 genome, produced resistance to amiloride equal to that of the amiloride passaged isolate (A3) in multiple replication cycles, indicating that no other mutations, including any unidentified mutations outside of the genomic region sequenced, were necessary to produce the resistant phenotype. The mutations conferred resistance not only to amiloride, but also to EIPA, confirming that amiloride and EIPA have the same mechanism of action. Serine 299 resides within the structural motif B of the catalytic palm domain of 3Dpol (Appleby et al., J. Virol. 79:277-288 2005; Hansen et al., Structure 5:1109-1122, 1997; Love et al., Structure 12:1533-1544, 2004; Thompson et al., EMBO J., 23:3462-3471, 2004). It is adjacent to N298 (N297 in poliovirus), which is located in the ribose-binding pocket of the polymerase active site and plays a crucial role in the selection of rNTPs over dNTPs (Gohara et al., Biochemistry 43:5149-5158, 2004; Gohara et al., J. Biol. Chem. 275:25523-25532, 2000; and Korneeva et al., J. Biol. Chem. 232:16135-16145, 2007). A372 resides within the structural motif E of 3Dpol, which has been implicated in helping to position the 3′ end of 5 the primer strand during RNA elongation. The location of both S299T and A372V mutations within structural motifs involved in the catalytic activity indicates that amiloride and EIPA bind within or close to the active site of the 3Dpol. S299 is highly conserved within the Enterovirus genus, with only 5% of 211 analysed isolates having a threonine at the structurally homologous position. In contrast, alanine is less prevalent (14%) than valine (86%) at the position corresponding to A372 of CVB3 Nancy 3Dpol. HRV2 3Dpol has both threonine and valine in positions corresponding to S299 and A372 of CVB3 3Dpol which may explain the lack of inhibition of HRV2 RNA replication by amiloride and EIPA. The 2A-D48G mutation had only minimal effects upon amiloride-resistance in multiple replication cycles, which is surprising because this mutation was present in four out of six amiloride-resistant isolates, and D48 is a highly conserved amino acid within the enteroviruses (present in 96% of 211 isolates; glycine has not been reported at this position). This mutation did, however, appear to have a more pronounced effect in a single replication cycle. The combination of 3D-A372V and 2A-D48G mutations produced a virus that replicated in the presence of amiloride or EIPA to a higher titre than the combined titres of the single mutants. This implies a synergistic effect of both mutations in a single replication cycle.

The amiloride derivatives have been shown to inhibit ion channels formed by transmembrane proteins of human immunodeficiency virus, hepatitis C virus, coronavirus and dengue viruses. HMA has been shown to inhibit HIV-1 replication in cultured macrophages and coronavirus replication in L929 cells when used at concentrations similar to those effective against CVB3 in this study; the effect attributed to inhibition of the ion channels formed by the Vpu or E proteins, respectively. The present data represent the first example of antiviral activity of amiloride derivates not due to inhibition of a viral ion channel. The location of mutations conferring CVB3 resistance to amiloride and EIPA, close to the active centre of CVB3 polymerase, indicates that these compounds may act as non-nucleoside polymerase inhibitors, a novel mechanism of activity for these compounds.

Together, these results demonstrate that amiloride and its functional derivatives can directly inhibit RNA-dependent, RNA polymerase of picornaviruses, and thus represent the first example of a non-nucleoside inhibitor of this enzyme for this family. As will be known to the skilled addressee, non-nucleoside inhibitors of other viral nucleic acid polymerases, such as the non-nucleoside drugs including Efavirenz and Delavirdine that are active against HIV reverse transcriptase, are a valuable component of the effective drug treatments against progression of HIV/AIDS.

The identification of critical amino acids in the RdRP, combined with knowledge of the three dimensional structure of the protein by analogy and modelling with the related poliovirus RdRP (see for example Thompson et al., 2005 (supra)), provides the basis for in silico docking studies to identify further antiviral compounds with chemical structures that do not share any significant similarity with amiloride, and to assist in the design of new chemical entities and derivatives of amiloride that demonstrate enhanced binding and thus enhanced antiviral potency against the picornaviruses.

Test compounds may be evaluated “in silico” for their ability to bind to RdRP prior to its actual synthesis and testing. In this manner, synthesis of inoperative compounds may be avoided. The quality of the fit of such entities to binding sites may be assessed by for example shape complementarity, or by estimating the energy of the interaction. (Meng et al., J. Comp. Chem., 13:505-524, 1992).

The design of chemical entities that associate with or antagonise RdRP generally involves consideration of two factors. First, the compound must be capable of physically and structurally associating with RdRP. Non-covalent molecular interactions important in the association of RdRP with its substrate include hydrogen bonding, van der Waals and hydrophobic interactions. Second, the compound must be able to assume a conformation that allows it to associate with RdRP. Although certain portions of the compound will not directly participate in this association with RdRP, those portions may still influence the overall conformation of the molecule. Such conformational requirements include the overall three-dimensional structure and orientation of the chemical entity or compound in relation to all or a portion of the active site, or the spacing between functional groups of a compound comprising several chemical entities that directly interact with RdRP.

Once an RdRP binding compound has been optimally selected or designed, as described above, substitutions may then be made in some of its atoms or side groups in order to improve or modify its binding properties. Generally, initial substitutions are conservative, i.e. the replacement group will have approximately the same size, shape, hydrophobicity and charge as the original group. It should of course, be understood that components known in the art to alter conformation should be avoided. Such substituted chemical compounds may then be analysed for the efficiency of fit of RdRP.

Putative RdRP-binding agents may be computationally evaluated and designed by means of a series of steps in which chemical entities or fragments are screened and selected for their ability to associate with the one or more binding sites of RdRP. This process may begin by visual inspection of the binding site on a computer screen based on structural coordinates. Selected fragments or chemical entities may then be positioned in a variety of orientations, or “docked,” to one or more RdRP interacting or active sites as defined herein. Docking may be accomplished using software, such as QUANTA and SYBYL, followed by energy minimization and molecular dynamics with standard molecular mechanics force fields, such as CHARMM or AMBER. Specialised computer programs may be of use for selecting interesting fragments or chemical entities. These programs include, e.g., GRID (Oxford University, Oxford, UK), MCSS (Molecular Simulations, USA), AUTODOCK (Scripps Research Institute, USA), DOCK (University of California, USA), XSITE (University College of London, UK) and CATALYST (Accelrys).

Useful programs to aid the skilled addressee in connecting chemical entities or fragments include CAVEAT (University of California, USA), 3D database systems and HOOK (Molecular Simulations, USA). De novo ligand design methods include those described in LUDI (Molecular Simulations, USA), LEGEND (Molecular Simulations, USA), LeapFrog (Tripos Inc.), SPROUT (University of Leeds, UK) and the like.

In a preferred embodiment, RdRP from picornavirus genera that cause significant infection in man are modelled upon the three-dimensional poliovirus RdRP or other RdRPs in order to test compounds for fit and efficacy.

Structure-based ligand design is well known in the art, and various strategies are available that can build on the present structural information to determine ligands that effectively modulate RdRP activity, for example, by binding the active site of RdRP or by competing with RdRP for binding by a ligand. Molecular modelling techniques include those described by Cohen et al., J. Med. Chem., 33:883-894, 1990, and Navia et al., Current Opinions in Structural Biology, 2:202-210, 1992.

Molecular modelling methods known in the art and as described herein may be used to identify an active site or binding pocket of RdRP or a variant including mutants thereof. Specifically, the structural coordinates provided by the present invention are used to characterise a three-dimensional structure of the RdRP molecule, molecular complex or an RdRP. From such a structure, putative active sites may be computationally visualised, identified and characterised based on the surface structure of the molecule, surface charge, steric arrangement, the presence of reactive amino acid residues, regions of hydrophobicity or hydrophilicity. Such putative active sites may be further refined using, for example, competitive and non-competitive inhibition assays, polymerase activity assays and/or by the generation and characterisation of RdRP or ligand mutants to identify critical residues or characteristics of the active site as described herein.

The three-dimensional representation or structure of at least a portion of a polypeptide of interest (eg. RdRP or its structurally similar variant) is understood to mean a portion of the three-dimensional surface structure or region of that polypeptide, including charge distribution and hydrophilicity/hydrophobicity characteristics, formed by at least three, or more, preferably at least three to ten, and even more preferably at least ten contiguous amino acid residues of the polypeptide. The contiguous residues forming such a portion may be residues that form a contiguous portion of the primary structure of the polypeptide or residues that form a contiguous protein of the three-dimensional surface of the polypeptide. Thus, the residues forming a portion of the three-dimensional structure of the polypeptide need not be contiguous in the primary sequence of the polypeptide but, rather, must form a contiguous portion of the polypeptide's surface. In a preferred embodiment, a portion of RdRP comprises or defines at least one RdRP interacting site/binding pocket as described therein.

The crystal structure of CVB3 is described in Jabafi et al., Acta Crystallograph 1:63 (Pt6):495-498, 2007. The amino acid sequence is 98% identical to polio 3D protein and the crystal structure of CVB3 and amiloride resistant and other variants thereof and other structurally related picornavirus RdRPs are determined by a number of different approaches. The present invention employs methods for determining the structure of a molecule or molecular complex whose structure is unknown, comprising the steps of obtaining a solution of the molecule or molecular complex whose structure is unknown and then generating X-ray crystallographic data from a crystal of the molecule or molecular complex. The X-ray crystallographic data from the molecule or molecular complex whose structure is unknown is then compared to the three-dimensional structure data obtained from a known RdRP structure of the present invention. Molecular replacement may be used to search for the optimal alignment of the RdRP structure of the present invention with X-ray diffraction data from crystals of the unknown molecule or molecular complex.

The present invention further provides that the structural coordinates of the present invention may be used with standard homology modelling techniques in order to predict the structure of the unknown three-dimensional structure or molecular complex. Homology modelling involves constructing a model of an unknown structure using structural coordinates of one or more related protein molecules, molecular complexes or parts thereof (i.e. active sites).

Homology modelling may be conducted by fitting common or homologous portions of the protein whose three-dimensional structure is to be solved, to the three-dimensional structure of homologous structural elements in the known molecule, specifically using the relevant (i.e. homologous) structural coordinates. Homology may be determined using amino acid sequence identity, homologous secondary structure elements and/or homologous tertiary folds. Homology modelling can include rebuilding part or all of a three-dimensional structure with replacement of amino acid residues (or other components) by those of the related structure to be solved.

Accordingly, a three-dimensional structure for the unknown molecule or molecular complex may be generated using the three-dimensional structure of the known RdRP molecule, refined using a number of techniques well known in the art and then used in the same fashion as the structural coordinates of the present invention, for instance, in applications involving molecular replacement analysis, homology modelling and rational drug design. Using such a three-dimensional structure, researchers identify putative binding sites and then identify or design agents to interact with these binding sites. These agents are then screened for an inhibitory effect upon the target molecule. In this manner, not only is the number of agents to be screened for the desired activity greatly reduced, but the mechanism of action on the target compound is better understood.

The skilled artisan will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Summary of sequence identifiers

| SEQUENCE | |
|---|---|
| ID NO: | DESCRIPTION |
| 1 | Amino acid sequence of RdRP of poliovirus type 1 |
| 2 | Amino acid sequence of RdRP of coxsackievirusB3 |
| | (Nancy, PO3313) |

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, |
| | Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

TABLE 3
Exemplary and Preferred Amino Acid Substitutions

| | EXEMPLARY | PREFERRED |
|---|---|---|
| Original Residue | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, | Leu |
| | Phe, Norleu | |
| Leu | Norleu, Ile, Val, | Ile |
| | Met, Ala, Phe | |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, | Leu |
| | Ala, Norleu | |

TABLE 4
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl)carbamylmethyl)glycine | Nnbhm | N-(N-(3,3-diphenylpropyl)carbamylmethyl)glycine | Nnbhe |
| 1-carboxy-1-(2,2-diphenyl- | Nmbc | | |
| ethylamino)cyclopropane | | | |

TABLE 5
Amino Acid Abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalamine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 6

| Genus | Virus name (synonym) followed by (acronym) |
|---|---|
| Enterovirus | bovine enterovirus 1 (BEV-1) |
| | bovine enterovirus 2 (BEV-2) |
| | human coxsackievirus A 1 to 22 (CAV-1 to 22) |
| | human coxsackievirus A 24 (CAV-24) |
| | human coxsackievirus B 1 to 6 (CBV-1 to 6) |
| | human echovirus 1 to 7 (EV-1 to 7) |
| | human echovirus 9 (EV-9) |
| | human echovirus 11 to 27 (EV-11 to 27) |
| | human echovirus 29 to 33 (EV-29 to 33) |
| | human enterovirus 68 to 71 (HEV68 to 71) |
| | human poliovirus 1 (HPV-1) |
| | human poliovirus 2 (HPV-2) |
| | human poliovirus 3 (HPV-3) |
| | porcine enterovirus 1 to 11 (PEV-1 to 11) |
| | simian enterovirus 1 to 18 (SEV-1 to 18) |
| | Vilyuisk virus |
| Rhinovirus | bovine rhinovirus 1 (BRV-1) |
| | bovine rhinovirus 2 (BRV-2) |
| | bovine rhinovirus 3 (BRV-3) |
| | human rhinovirus 1A (HRV-1A) |
| | human rhinovirus 1 to 100 (HRV-1 to 100) |
| Hepatovirus | hepatitis A virus (HAV) |
| | simian hepatitis A virus (SHAV) |
| Cardiovirus | encephalomyocarditis virus (EMCV) |
| | (Columbia SK virus); (mengovirus) |
| | (mouse Elberfield virus) |
| | Theiler's murine encephalomyelitis virus (TMEV) |
| | (murine poliovirus) |
| Aphthovirus | foot-and-mouth disease virus A (FMDV-A) |
| | foot-and-mouth disease virus ASIA 1 (FMDV-ASIA1) |
| | foot-and-mouth disease virus C (FMDV-C) |
| | foot-and-mouth disease virus O (FMDV-O) |
| | foot-and-mouth disease virus SAT 1 (FMDV-SAT1) |
| | foot-and-mouth disease virus SAT 2 (FMDV-SAT2) |
| | foot-and-mouth disease virus SAT 3 (FMDV-SAT3) |
| Parechovirus | Human parechovirus |
| Erbovirus | Equine rhinitis B virus |
| Kobovirus | Aichi virus |
| Teschovirus | Porcine teschovirus |

TABLE 7

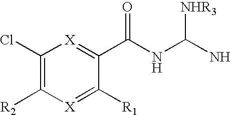

| | | | | | IC₅ | CC₅ |
|---|---|---|---|---|---|---|
| Name | X | R₁ | R₂ | R₃ | μM | μM |
| EIPA | N | NH₂ | N(Et)CHMe₂ | H | 2 | 25 |
| MIBA | N | NH₂ | N(Me)CH₂CHMe₂ | H | 2 | 25 |
| HMA | H | NH₂ | 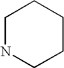 | H | 2 | 25 |
| CHPG | CH | OH | H | C₂H₄Ph | 2 | 15 |
| CHG | CH | OH | H | H | 5 | 90 |
| 2,4-DCB | N | NH₂ | NH₂ | 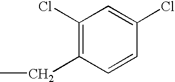 | 5 | 14 |
| 3,4-DCB | N | NH₂ | NH₂ | 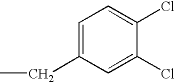 | 5 | 14 |
| CHMG | CH | OH | H | CH₃ | 9 | 155 |
| Benzamil | N | NH₂ | NH₂ | CH₂Ph | 10 | 100 |
| CMPG | CH | OCH₃ | H | C₅H₁ | 28 | 40 |
| Amiloride | N | NH₂ | NH₂ | H | 50 | >1000 |
| DMA | N | NH₂ | NMe₂ | H | 90 | 190 |
| CMG | CH | OCH₃ | H | H | 110 | 350 |

### BIBLIOGRAPHY

Altschul et al., Nucleic Acids Res., 25:3389-3402, 1997.
Appleby et al., J. Virol. 79:277-288 2005.
Ausubel et al., Current Protocols in Molecular Biology John Wiley & Sons Inc, 1994-1998, Chapter 15.
Ausubel (Ed) Current Protocols in Molecular Biology, 5^{th} Edition, John Wiley & Sons, Inc, NY, 2002.
Beddell, Chem. Soc. Reviews: 279, 1985.
Bonner et al., Eur. J. Biochem., 46:83, 1974.
Cohen et al., J. Med. Chem., 33:883-894, 1990.
Dayhoff et al., Atlas of Protein Sequence and Structure, Natl. Biomed. Res. Found., Washington, D.C., 1978.
Erickson et al., Science, 249:527-533, 1990.
Gazina et al., Antiviral Res. 67:98-106, 2005.
Gonnet et al., 1992, Science, 256(5062):1443-1445, 1992.
Goodford, J. Med. Chem., 27:557, 1984.
Gohara et al., J. Biol. Chem. 275:25523-25532, 2000.
Gohara et al., Biochemistry 43:5149-5158, 2004
Guner (Ed), Pharmacophore perception, development, and use in drug design, International University Line, 2000.
Guthridge et al, Stem Cells, 16:301, 1998.
Hansen et al., Structure 5:1109-1122, 1997
Hodgson, Bio/Technology, 9:19-21, 1991.
Hol, Angew. Chem., 25:767, 1986.
Jabafi et al., Acta Crystallograph 1:63 (Pt6):495-498, 2007.
Korneeva et al., J. Biol. Chem. 282:16135-16145, 2007
Kuntz et al, J. Mol. Biol., 161:269-288, 1982.
Kurrek, Eur. J. Biochem., 270:1628-1644, 2003.
Lahser et al. Analytical Biochemistry 325:247-245, 2004.
Langer et al. (Eds), Pharmacophores and pharmacophore searches, John Wiley & Sons, Inc, NY, 2006.
Love et al., Structure 12:1533-1544, 2004.
Marmur et al., J. Mol. Biol., 5:109, 1962.
Martin et al. (Eds), Designing bioactive molecules: Three-dimensional techniques and applications, American Chemical Society, NY, 1998.
Meng et al., J. Comp. Chem., 13:505-524, 1992.
Navia et al., Current Opinions in Structural Biology, 2:202-210, 1992.
Noren et al, Science, 244:182-188, 1989.
Reddy et al. (Eds), Free energy calculations in rational drug design, Springer-Verlag, 2001.
Remington's Pharmaceutical Sciences, 18^{th} Ed., 1990, Mack Publishing, Company, Easton, Pa., U.S.A.
Sambrook, Molecular Cloning: A Laboratory Manual, 3^{rd} Edition, CSHLP, CSH, NY, 2001.
Schaeffer et al., Nucleic Acids Res., 29:2994-3005, 2001.
Sheridan et al., Acc. Chem. Res., 20:322, 1987.
Summerton et al., Antisense and Nucleic acid Drug Development, 7:187-195, 1997.
Thompson et al., EMBO J., 23:3462-3471, 2004.
van Ooij et al., J. Gen. Virol. 87:103-113, 2006.
Verlinde, W. G. J. Structure, 2:677, 1994.
Wells, Methods Enzymol., 202:2699-2705, 1991.
Wermuth (Ed), The practice of medicinal chemistry, 2^{nd} Edition, Academic Press, NY, 2003.
Wessels et al. J. Virol. 79:5163-5173, 2005.
Zubay, G., Biochemistry, 3^{rd} edition, Wm.C. Brown Publishers, 1993.

1. A method of anti-viral drug design or testing comprising the use of structural coordinates comprising an interacting site of a viral RNA dependent RNA polymerase (RdRP) and/or a variant thereof and/or a viral RNA dependent RNA polymerase (RdRP) activity assay to evaluate the anti-viral activity of an amiloride-like compound.

2. The method of claim 1 wherein the activity assay evaluates RdRP binding or enzymatic activity.

3. The method of claim 1 wherein the RdRP is an enterovirus RdRP.

4. A method of evaluating the ability of an amiloride-like compound to modulate viral activity wherein said method comprises: computationally generating a three dimensional molecular representation comprising at least one interacting site of a viral RdRP and/or a variant thereof; computationally generating a three dimensional molecular representation of the test amiloride-like compound; performing a molecular fitting (docking) operation; computationally quantifying the association between the RdRP and/or a variant thereof and the test compound based on the output of the fitting (docking) operation.

5. The method of claim 4 wherein the molecular representation includes an interacting site of a viral RdRP bound to GTP.

6. The method of claim 4 wherein the molecular representation includes an interacting site of a viral RdRP bound to NTP.

7. The method of claim 4 wherein the interacting site comprises one or more of a palm domain and a finger domain (pinky, middle, ring, index and/or thumb).

8. The method of claim 7 wherein the palm domain comprises one or more or consists of polymerases domains including: motif A (aa225-240 of 3D of poliovirus or corresponding amino acids from other Picornaviridae); motif B (aa290-312 or corresponding amino acids from other Picornaviridae); motif C (aa318-336 or corresponding amino acids from other Picornaviridae); motif D (339-354 or corresponding amino acids from other Picornaviridae); motif E (aa369-380 of 3D of poliovirus, aa370-381 of CVB3 or corresponding amino acids from other Picornaviridae).

9. The method of claim 4 wherein the interacting site of RdRP comprises an NTP-binding centre and/or an E motif.

10. The method of claim 4 wherein the three dimensional molecular representation of RdRP consists essentially of an NTP-binding centre and/or an E motif.

11. (canceled)

12. (canceled)

13. A method for identifying a compound which inhibits RdRP activity, the method comprising contacting in silico or in vitro an RdRP and/or a variant thereof with an amiloride-like compound and determining whether or not an activity of RdRP is decreased in the presence of the amiloride-like compound.

14. The method of claim 13 wherein the activity is RdRP binding or RdRP enzymatic activity.

15. A method for identifying a compound which inhibits RdRP activity, the method comprising contacting an RdRP and/or variant thereof with a competitor amiloride-like compound wherein said competitor comprises a detectable label, whereby said competitor binds to RdRP and/or a variant thereof and is capable of being displaced by an inhibitor.

16. The method of any one of claim 1, 4, 13 or 15 wherein the RdRP is an enterovirus RdRP and/or a variant thereof.

17. The method of claim 16 wherein the enterovirus is poliovirus or coxsackievirus.

18. The method of any one of claim 1, 4, 13 or 15 wherein the RdRP variant is an amiloride-resistant mutant form of RdRP.

19. The method according to any one of claim 1, 4, 13 or 15 wherein the amiloride-like compound is selected from the group consisting of amiloride, EIPA, Benzamil, HMA or a derivative or variant thereof.

20. An amiloride-resistant CVB3 variant.

21. The amiloride-resistant variant of claim 20 comprising at least one or two or more RdRP mutations including S299T, A372V and/or D48G.
